(19) United States
(12) Patent Application Publication
(10) Pub. No.: US 2024/0136022 A1
(43) Pub. Date: Apr. 25, 2024

(54) METHODS AND COMPOSITIONS FOR DETECTING CANCER USING FRAGMENTOMICS

(71) Applicant: PetDx, Inc., La Jolla, CA (US)

(72) Inventors: Kristina Kruglyak, La Jolla, CA (US); Francesco Marass, La Jolla, CA (US); Wai Yi Tsui, La Jolla, CA (US)

(21) Appl. No.: 18/512,655

(22) Filed: Nov. 17, 2023

Related U.S. Application Data
(63) Continuation of application No. PCT/US2022/030301, filed on May 20, 2022.
(60) Provisional application No. 63/202,006, filed on May 21, 2021.

(51) Int. Cl. G16B 40/20; G16B 20/00

(52) U.S. Cl. CPC G16B 40/20; G16B 20/00

(57) ABSTRACT

Provided herein are methods and kits for measuring fragment size distribution of DNA fragments from a sample of a subject, for the purposes of cancer or tumor detection, characterization, and/or management.

### CROSS REFERENCE TO RELATED APPLICATION

This application is a continuation of PCT Application No. PCT/US2022/030301, filed May 20, 2022, which claims the benefit of U.S. Provisional Application No. 63/202,006, filed May 21, 2021, each of which is hereby incorporated by reference in its entirety.

### FIELD

The present disclosure relates to methods for detecting, characterizing, or managing cancer or a tumor in a subject by analyzing a fragment size distribution of the DNA fragments from a sample.

### BACKGROUND

Companion animals, such as dogs and cats are enjoying longer lifespans as veterinary medicine continues to improve. However, this increased lifespan has led to a higher rate of cancers among companion animals. By some estimates, over 50% of dogs over ten years of age are going to die from a cancer-related health issue. Cats are also susceptible to a variety of cancers. Among the most common cancers in these animals are lymphoma, squamous cell carcinoma (skin cancer), mammary cancer, mast cell tumors, oral tumors, fibrosarcoma (soft tissue cancer), osteosarcoma (bone cancer), respiratory carcinoma, intestinal adenocarcinoma, and pancreatic/liver adenocarcinoma.

Certain breeds of cats are more prone to certain cancers than others. Signs and symptoms differ depending on the type and stage of the cancer. Unfortunately, detection and diagnosis of these cancers is often difficult, and invasive biopsy tests usually need to be performed to make an accurate diagnosis.

The situation is similar for dogs. Certain canine breeds are known to be susceptible to particular cancers. Rafalko, BIORXIV, 2022. For example, larger dogs are more susceptible to developing osteosarcoma. German Shepherds, Golden Retrievers, Labrador Retrievers, Pointers, Boxers, English Settlers, Great Danes, Poodles, and Siberian Huskies are susceptible to developing hemangiosarcoma (HSA). HSA tends to affect large breed animals more often than smaller ones.

Current methods of cancer diagnosis include imaging, radiolabeling, and biopsies. Liquid biopsies offer diagnostic information that is otherwise only accessible through invasive biopsies. The first applications of liquid biopsies are based on the detection of genetic markers such as sex differences, genetic polymorphisms, or mutations. Noninvasive prenatal testing has been used globally for the screening of fetal chromosomal aneuploidies and has led to a considerable reduction in invasive prenatal testing, such as use of amniocentesis. Liquid biopsies for organ transplant patients have been used to monitor graft dysfunction. Cancer liquid biopsies have been used for the selection of targeted therapies and monitoring of disease progression. However, currently available techniques with biopsies do not provide a relatively inexpensive and simple way to perform cancer or tumor detection.

### SUMMARY

Described herein are methods and compositions for measuring fragment size distribution of DNA obtained from a sample from a subject. In some embodiments, the compositions and methods are used for the detection, diagnosis, and screening of cancer in subjects.

Some embodiments provided herein relate to methods of detecting a cancer or tumor in a subject. In some embodiments, the methods include isolating a circulating cell free DNA (cfDNA) sample from the subject, sequencing the cfDNA sample to measuring one or more fragment size distribution, comparing the one or more fragment size distribution to a second fragment size distribution, wherein the second fragment size distribution is obtained from one or more control subject, and determining the presence of the cancer or tumor based upon the comparisons of the two distributions. In some embodiments, the one or more subjects include the same subject or one or more healthy subjects. In some embodiments, the sequencing of the cfDNA sample is whole genome sequencing or next generation sequencing.

In some embodiments, the subject is mammalian. In some embodiments, the subject is canine, feline, equine, or human. In some embodiments, the cfDNA sample is isolated from the blood of the subject. In some embodiments, the blood of the subject further includes circulating tumor DNA (ctDNA). In some embodiments, the cancer is a hematological cancer. In some embodiments, the cancer is a lymphoma.

In some embodiments, the methods further include creating a model of the one or more fragment size distribution. In some embodiments, the model of the one or more fragment size distribution is a statistical model. In some embodiments, the model of the one or more fragment size distribution is obtained from one or more features extracted from the one or more fragment size distribution. In some embodiments, the one or more features include median, mean, area under the curve (AUC), amplitude of oscillations, variance, standard deviations, length intervals, or a combination thereof.

In some embodiments, the methods further include classifying samples as tumor or normal based on the one or more features. In some embodiments, the model of the second fragment size distribution is a statistical model. In some embodiments, comparing the one or more fragment size distribution to the second fragment size distribution is performed through KL divergence. In some embodiments, the one or more fragment size distribution is calculated from at least one of length or sequence of cfDNA fragments in the sample. In some embodiments, the second fragment size distribution is a baseline fragment size distribution.

In some embodiments, the methods further include ligating adapters to the isolated cfDNA and using a universal primer to target the adapters to generate amplified fragments. In some embodiments, the one or more fragment size distribution is measured by determining a number and distribution of amplified fragment sizes using whole genome sequencing or next generation sequencing. In some embodiments, comparing the one or more fragment size distribution to the second fragment size distribution is performed by comparing the number and distribution of the amplified fragment sizes to one or more healthy subjects to determine if the number and distribution of the amplified fragment sizes in the subject differs from the number and distribution of the amplified fragment sizes in the one or more healthy subjects. In some embodiments, the universal primer further includes a sequence specific primer. In some embodiments, a statistically significant difference between the one or more fragment size distribution in the subject and the second fragment size distribution in the one or more healthy subjects indicates the presence of a cancer or tumor. In some embodiments, a non-statistically significant difference between the one or more fragment size distribution in the subject and the second fragment size distribution in the one or more healthy subjects indicates the lack of presence of a cancer or tumor.

Some embodiments provided herein relate to methods of predicting a cancer signal of origin (CSO) from a subject having a positive cancer detected signal. In some embodiments, the methods include isolating a circulating cell free DNA (cfDNA) sample from the subject, sequencing the cfDNA sample to determine a fragment size distribution and a copy number (CN) profile, obtaining a positive cancer signal detected from the CN profile, comparing the fragment size distribution in CN amplified and/or depleted regions to a control CN region, and predicting the CSO based on the difference or lack thereof between the fragment size distribution of the CN amplified and/or depleted regions and the control CN region. In some embodiments, the lack of difference between the fragment size distribution of the CN amplified and/or depleted regions and the control CN region is a prediction for hematological cancer.

Some embodiments provided herein relate to methods of detecting a cancer or tumor in a subject. In some embodiments, the methods include isolating a circulating cell free DNA (cfDNA) sample from the subject, sequencing the cfDNA sample to determine a one or more fragment size distribution, generating an experimental model of the one or more fragment size distribution, comparing the one or more fragment size distribution to a second fragment size distribution from one or more control subjects, and determining the presence of the cancer or tumor based upon the comparisons of the two distributions. In some embodiments, the one or more control subjects include the same subject or one or more healthy subjects. In some embodiments, the experimental model of the one or more fragment size distribution is a statistical model. In some embodiments, the experimental model of the one or more fragment size distribution is obtained from one or more features extracted from the one or more fragment size distribution. In some embodiments, the one or more features include mean, area under the curve (AUC), amplitude of oscillations, standard deviations, length intervals, or a combination thereof.

In some embodiments, the methods further include comparing the experimental model obtained from the cfDNA sample to a control model obtained from a control cfDNA sample in an individual known to not have cancer or a tumor. In some embodiments, the likelihood for the subject having a cancer or tumor is determined by comparing the experimental model to the control model. In some embodiments, the likelihood for the subject having a cancer or tumor is determined by comparing one or more features of the experimental model to one or more features of the control model. In some embodiments, comparing the one or more fragment size distribution to the second fragment size distribution from at least one healthy subject is conducted through KL divergence.

Some embodiments provided herein relate to methods of measuring fragment size distribution in a sample. In some embodiments, the method include isolating a DNA sample from a subject, sequencing the DNA sample to determine a fragment size distribution, measuring one or more features from the fragment size distribution, and generating an experimental model of the fragment size distribution. In some embodiments, the subject has or is suspected of having cancer. In some embodiments, the experimental model is a statistical model. In some embodiments, the experimental model is obtained from the one or more features. In some embodiments, the one or more features include mean, area under the curve (AUC), amplitude of oscillations, standard deviations, length intervals, or a combination thereof.

In some embodiments, the methods further include identifying the sample as a tumor sample or as a normal sample based on the one or more features. In some embodiments, the fragment size distribution is calculated from at least one of length or sequence of DNA fragments in the sample. In some embodiments, the DNA sample is a cell free DNA (cfDNA) sample. In some embodiments, the DNA sample is isolated from blood of the subject. In some embodiments, the blood further includes circulating tumor DNA (ctDNA). In some embodiments, the sequencing includes whole genome sequencing or next generation sequencing. In some embodiments, the methods further include ligating adapters to the isolated DNA and using a universal primer to target the adapters to generate amplified fragments. In some embodiments, the one or more fragment size distribution is measured by determining a number and distribution of amplified fragment sizes using whole genome sequencing or next generation sequencing. In some embodiments, the universal primer further includes a sequence specific primer.

### BRIEF DESCRIPTION OF THE DRAWINGS

FIG. 1 is a line graph which shows an exemplary profile of the average density of cfDNAs with a particular fragment length across cfDNA samples taken from normal, healthy subjects.

FIGS. 2A-2C show line graphs of exemplary conversion of a fragment size distribution into a negative binomial mixture model (FIG. 2A), Gaussian mixture model (FIG. 2B), and naïve mixture model (FIG. 2C). In each figure, the grey line is a sample, and the black line is a model fit of the sample. In FIG. 2C, the grey line is a sample, and the circles denote the locations and heights of each identified peak.

FIGS. 3A-3C show point graphs of exemplary distribution of modes using the negative binomial mixture model (FIG. 3A), Gaussian mixture model (FIG. 3B), and naïve mixture model (FIG. 3C) on reversed data. Normal samples are either those from the baseline run (circles), or samples from the test run (here called ‘test-normal’) (triangles). “Mode3” shows the scaling used in the graph, wherein the higher mode values are reflected in larger circles or triangles.

FIGS. 4A-4B show point graphs of exemplary distribution of weights using the negative binomial mixture model (FIG. 4A) and Gaussian mixture model (FIG. 4B) on reversed data. Normal samples are either those from the baseline run (circles), or samples from the test run (here called ‘test-normal’) (triangles). Weights” are the proportions of each component (nucleosome peak) of the mixture model. “Weight3” shows the scaling used in the graph, wherein the higher weight values are reflected in larger circles or triangles.

FIGS. 5A-5B show point graphs of exemplary distribution of scales using the negative binomial mixture model (FIG. 5A) or Gaussian mixture model (FIG. 5B) on reversed data. Normal samples are either those from the baseline run (circles), or samples from the test run (here called ‘test-normal’) (triangles). “Scales” for the negative binomial mixture model on reversed data is the overdispersion, i.e. small values cause more variance. “Scale3” shows the scaling used in the graph, wherein the higher scale values are reflected in larger circles or triangles.

FIGS. 6A-6B show point graphs of exemplary principal component analysis (PCA) using the negative binomial mixture model (FIG. 6A) or Gaussian mixture model (FIG. 6B) on reversed data. Normal samples are either those from the baseline run (circles), or samples from the test run (here called ‘test-normal’) (triangles). The extracted features do not separate samples by test. Almost all variation is captured in one principal component. “PC3” shows the scaling used in the graph, wherein the higher principal component values are reflected in larger circles or triangles.

FIG. 7 is a point graph which shows a PCA plot of the normalized fragment length data comparing PC values across Batches 1, 2, and 3. “PC3” shows the scaling used in the graph, wherein the higher principal component values are reflected in larger circles.

FIGS. 8A-8D show a boxplot (also called a box and whiskers graph) of the PC values by batch across all samples (FIG. 8A), non-normal samples (FIG. 8B), normal samples (FIG. 8C), and baseline samples (FIG. 8D) for Batches 1, 2, and 3. Baseline samples are a subset of the normal samples disclosed herein.

FIGS. 9A-9B show line graphs of exemplary profiles of the density of cfDNAs with a particular fragment length across Batch 1, 2, and 3 of cfDNA samples taken from normal subjects (FIG. 9A) and baseline normal subjects (FIG. 9B).

FIGS. 10A-10B show point graphs of exemplary comparison of peak proportions using the initial set of statistics by creating the combined normals from all normal samples across Batches 1-3 (FIG. 10A) and baseline normals in the construction of the combined normal sample (FIG. 10B). “Peak3” shows the scaling used in the graph, wherein the higher peak values are reflected in larger circles.

FIGS. 11A-11B show point graphs of exemplary plot of oscillation values (FIG. 11A) and AUC values (FIG. 11B) across Batch 1, 2, and 3, separated by baseline, non-normal, and normal groups.

FIG. 12 shows a boxplot of age distribution of subjects separated by batch across Batch 1, 2, and 3.

FIG. 13 is a point graph which depicts the KL divergence values of samples separated into baseline, normal, and tumor groups.

FIG. 14 is a point graph which depicts the KL divergence values of samples from batches 4-7 and 12 grouped into normal, and tumor groups.

FIG. 15 is a graph which shows the correlations between extracted features (mean, AUC, oscillations, and standard deviations) according to the Gaussian mixture model. The parameters of these distributions were estimated by Markov chain Monte Carlo. Means, SDs, and weights were obtained from the mixture model for all samples; short fragments' AUC are relative to the first mode of each sample; the oscillations were computed from crests and troughs as identified in the baseline samples.

FIGS. 16A-16D show a distribution of accuracy, sensitivity, specificity, PPV, and F-1 scores computed for each threshold using a probabilistic approach, then optimized for: specificity (FIG. 16A), F-1 scores (FIG. 16B), PPV scores (FIG. 16C), and sensitivity (FIG. 16D).

FIG. 17 shows a profile of the differences in fragment lengths between the normalized counts of the average normal sample and the average tumor sample.

FIG. 18 shows a profile of the average normalized counts of cfDNAs with a particular fragment length across batches 1-3 for either normal or tumor samples.

FIG. 19 shows a PCA analysis of all samples in batches 1-3, and the 2D density contour of normal samples. “PC3” shows the scaling used in the graph, wherein the higher principal component values are reflected in larger circles.

FIG. 20 shows a dot plot of the KL divergence values from mean of baseline, normal, and tumor samples.

FIG. 21 shows a dot plot of the KL divergence values from mean of baseline, normal, and tumor samples after removing two outlier samples from the baseline mixture model.

FIG. 22 shows a graph plotting the prior distribution of tumor content as a function of the tumor content value.

FIG. 23A shows a graph plotting the inferred tumor content versus the expected tumor content for sample 201-20885 mixed into healthy cfDNA.

FIG. 23B shows a graph plotting the inferred tumor content versus the expected tumor content for sample 201-00316 mixed into healthy cfDNA.

FIG. 24A shows a graph plotting the inferred tumor content versus the expected tumor content for sample 201-00015 mixed into healthy cfDNA.

FIG. 24B shows a graph plotting the inferred tumor content versus the expected tumor content for sample 301-30640 mixed into healthy cfDNA.

FIG. 25 shows the fragment length distributions of chromosomes that are lost, neutral, or gained, for sample 201-00015.

FIG. 26 shows the adjusted separation values of samples by cancer type. Tumor types with a single sample, and samples with no separation and low tumor content are not shown.

FIG. 27 shows a plot for the choice of threshold. Every threshold from 135 to 175 was tested in increments of one, then plotted as raw separation value versus threshold that produced the maximum separation.

FIG. 28 shows the effect of data smoothing on the choice of threshold, shown as a change in chosen threshold for samples with and without spline smoothing.

FIG. 29A shows the linear relationship between the separation values computed using loss-gain and neutral-gain (left panel) or loss-neutral (right panel) formulae of samples selected for having all three copy number (CN) groups. The read cutoff was at 0.

FIG. 29B shows the correlation between the residuals of the separation value correction and the minimum number of reads considered (M) between loss-gain and neutral-gain (left panel) or loss-neutral (right panel). The read cutoff was at 0.

FIG. 29C shows the linear relationship between separation values computed using loss-gain and neutral-gain (left panel) or loss-neutral (right panel) formulae after correction. The read cutoff was 200,000.

FIG. 30 shows the accuracy of the adjustment of the loss-neutral and neutral-gain formulas, plotted as the difference between the adjusted and the expected values.

FIG. 31 shows a plot of the reads per chromosome versus the average KL per chromosome per sample.

FIG. 32 shows the change in KL divergence by using fragmentomics by chromosome over a genome-wide approach. The solid horizontal lines represent possible thresholds.

FIG. 33 shows the predicted KL versus the true KL using chromosome-specific hyperbolae, with parameters learnt using model 6.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein. All references cited herein are expressly incorporated by reference herein in their entirety and for the specific disclosure referenced herein.

Embodiments relate to methods, systems, and compositions for screening subjects for their likelihood to have a cancer or a tumor. In some embodiments, a cancer or a tumor is screened for by isolating a circulating cell free DNA (cfDNA) sample from a subject, such as a canine, suspected of having a cancer or tumor, sequencing the cfDNA fragments in the sample, calculating a size distribution based upon at least one cfDNA fragment, creating a model or summary statistic of the fragment size distribution, comparing the model of the fragment size distribution to a second model derived from at least one healthy subject, and determining the presence of the cancer or tumor based upon the comparisons of the two models. The sequencing of the cfDNA can be performed through any method recognized by one skilled in the arts, such as targeted or genome-wide sequencing. Other non-limiting examples include methods using nanopores, emulsion, and ‘sequencing by binding’ cycled sequencing methods.

In some embodiments, a cancer or a tumor is screened for by the comparison of models. In some embodiments, these models are mixture models. Models are derived from the fragment size distribution profile of at least one fragment. “Fragment distribution” as used herein has its usual meaning as understood by those skilled in the art and thus refers to the length, sequence, fragmentation, and other distribution properties of an at least one DNA fragment taken from a cfDNA sample. A “fragment size distribution” is understood as a fragment distribution focusing on the size of fragments, including length or fragmentation. As disclosed herein, a model can be formed for the subject suspected of having a cancer or a tumor, as well as a model for one or more healthy subjects. These models can then be compared to one another to monitor for significant differences. Non-limiting examples of models include summary statistics, the number and shape of nucleosomal peaks, the proportion of fragments longer or shorter than a certain threshold, the proportion of fragments in certain intervals, the approximation of the data with statistical distributions, and discriminatory learning methods, such as support vector machines or neural networks. Non-limiting examples of detectable differences include the location of the peaks (mode), the height of the peaks (weight), the spread of the peaks (scale), the proportion of fragments longer or shorter than a certain threshold, the amplitude of oscillations, the overall shape of the fragment size distribution, Principal Component values, and Kullback-Leibler (KL) divergence between two models. In some embodiments, a statistically significant difference between the fragment size distribution in the subject suspected of having a cancer or tumor and the fragment size distribution in the one or more healthy subjects indicates the presence of a cancer or tumor. In some embodiments, a non-statistically significant difference between the fragment size distribution in the subject suspected of having a cancer or tumor and the fragment size distribution in the one or more healthy subjects indicates the lack of presence of a cancer or tumor.

A variety of ways exist for determining the fragment size distribution of the cfDNA within a subject. In one embodiment a blood sample is taken from a subject. Circulating free DNA (cfDNA) from the blood is obtained. In some embodiments, the blood sample comprises circulating tumor DNA (ctDNA). The cfDNA is isolated by removing blood cells from the sample so that only cfDNA remains in the sample. In some embodiments, a set of random PCR primers for whole genome sequencing are added to the sample to amplify the fragments while preserving their original fragment length within the sample.

Polymerase is then added to the mixture, so the primers are extended through the full length of each fragment. The amplified fragments may include sequencing ends which are formatted to be used within a Next Generation Sequencing (NGS) system to identify the nucleotide sequences in the fragments in one embodiment.

Methods and compositions provided herein improve the detection, diagnosis, staging, screening, treatment, and management of cancer in subjects, particularly in humans, mammals, and other types of subjects. As mentioned above, embodiments include identifying the fragment distribution of cfDNA circulating biological fluids, such as blood. In one embodiment, the nucleic acid sequence elements are found in circulating tumor DNA in the blood. In some embodiments, the nucleic acid sequence elements may be found in cell-free DNA, in saliva, or urine.

As used herein, “detecting” with respect to measuring a cancer or tumor includes the use of an instrument used to observe and record a signal corresponding to a level or measurement of cancer, or materials required to generate such a signal. In various embodiments, the detecting includes any suitable method, including amplification, sequencing, arrays, fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

Some embodiments provided herein relate to kits. In some embodiments, the kits are for determining cancer in a subject. In some embodiments, the kits include whole genome sequencing primers for amplifying cfDNA in a biological sample from a subject, and a polymerase for amplifying the primers.

It should be realized that the analysis described herein may be part of a larger diagnostic suite used to determine a subject's overall health. For example, the analysis of fragment size distributions of cfDNA in a subject may be used simultaneously or sequentially with other methods for detection, diagnosis, staging, screening, monitoring, treatment, and management of cancer including additional genetic variance analysis. These procedures may be useful to detect a variety of cancers, including leukemia, squamous cell carcinoma, feline mammary cancer, mast cell tumors, bladder cancer, osteosarcoma, hemangiosarcoma or a variety of other cancers afflicting subjects.

In some embodiments, the methods include obtaining or having obtained a biological sample from a subject that is suspected of having cancer. In some embodiments, the sample is a liquid biopsy sample, such as a blood sample. In some embodiments, the sample includes cfDNA. In some embodiments, the sample is provided in an amount of less than 10 mL, such as 10 mL, 9 mL, 8 mL, 7 mL, 6, mL, 5 mL, 4 mL, 3 mL 2 mL, 1 mL, 500 μL, 250 μL, 100 μL or an amount within a range defined by any two of the aforementioned values. In some embodiments, the sample includes DNA in an amount of less than or equal to 10 μg, such as 10 μg, 5 μg, 1 μg, 500 ng, 100 ng, 50 ng, 10 ng, 5 ng, 1 ng, 500 pg, 100 pg, 50 pg, 10 pg, 9, pg, 8 pg, 7 pg, 6 pg, 5 pg, 4 pg, 3 pg, 2 pg, or 1 pg, or in an amount within a range defined by any two of the aforementioned values. In some embodiments, the method includes purifying the DNA from the sample. Purifying the DNA may be accomplished using DNA purification techniques, including, for example extraction techniques, precipitations, chromatography, bead-based methods, or commercially available kits for DNA purification. In some embodiments, the methods can be used to determine the probable cancer type or cancer tissue of origin based on one or more of the fragment size distribution features.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

As used herein, “a” or “an” can mean one or more than one.

As used herein, the term “about” or “approximately” has its usual meaning as understood by those skilled in the art and thus indicates that a value includes the inherent variation of error for the method being employed to determine a value, or the variation that exists among multiple determinations.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm”.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “function” and “functional” as used herein have their plain and ordinary meaning as understood in light of the specification, and refer to a biological, enzymatic, or therapeutic function.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 81, 82, 83, 84, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, or living cells which make up these living organisms, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

As used herein, “nucleic acid”, “nucleic acid molecule”, or “nucleotide” refers to polynucleotides or oligonucleotides such as deoxyribonucleic acid (DNA) or ribonucleic acid (RNA), oligonucleotides, fragments generated by the polymerase chain reaction (PCR), and fragments generated by any of ligation, scission, endonuclease action, exonuclease action, and by synthetic generation. Nucleic acid molecules can be composed of monomers that are naturally occurring nucleotides (such as DNA and RNA), or analogs of naturally occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, pho sphoro seleno ate, pho sphorodiseleno ate, phosphoroanilothioate, phosphoranilidate, phosphoramidate, and the like. The term “nucleic acid molecule” also includes so-called “peptide nucleic acids,” which comprise naturally occurring or modified nucleic acid bases attached to a polyamide backbone. Nucleic acids can be either single stranded or double stranded.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The terms “DNA fragment” and “nucleic acid fragment” have their ordinary meaning as understood by those of skill in the art and refer to a polynucleotide sequence obtained from a genome at any point along the genome and encompassing any sequence of nucleotides.

The term “fragment size distribution” has its ordinary meaning as understood by those of skill in the art, and refers to information regarding one or more of: the total number of nucleic acid fragments present in a sample, the size of one or more nucleic acid fragments in the sample, the absolute or relative abundance levels of nucleic acid fragments of a specific size or size range, and the absolute or relative abundance levels of nucleic acid fragments of different size present in the sample.

The term “fragment size” has its ordinary meaning as understood by those of skill in the art, and as used herein in reference to a nucleic acid molecule, refers to the number of base pairs of the nucleic acid, and denotes the length of the molecule.

The term “gene” as used herein have their plain and ordinary meaning as understood in light of the specification, and generally refers to a portion of a nucleic acid that encodes a protein or functional RNA; however, the term may optionally encompass regulatory sequences. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs and miRNAs. In some cases, the gene includes regulatory sequences involved in transcription, or message production or composition. In other embodiments, the gene comprises transcribed sequences that encode for a protein, polypeptide, or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide, or peptide encoding sequences, etc. In this respect, the term “gene” is used for simplicity to refer to a nucleic acid comprising a nucleotide sequence that is transcribed, and the complement thereof. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express or may be adapted to express using nucleic acid manipulation technology, proteins, polypeptides, domains, peptides, fusion proteins, mutants and/or such like.

The terms “cancer” and “cancerous” have their ordinary meaning as understood in light of the specification and refer to or describe the physiological condition in animals that is typically characterized by unregulated cell growth. A “tumor” comprises one or more cancerous cells. In some embodiments, the tumor is a solid tumor. There are several main types of cancer. Carcinoma is a cancer that originates from epithelial cells, for example skin cells or lining of intestinal tract. Sarcoma is a cancer that originates from mesenchymal cells, for example bone, cartilage, fat, muscle, blood vessels, or other connective or supportive tissue. Leukemia is a cancer that originates in hematopoietic cells, such as the bone marrow, and causes large numbers of abnormal blood cells to be produced and enter the blood. Lymphoma and multiple myeloma are cancers that originate in the lymphoid cells of lymph nodes. Central nervous system cancers are cancers that originate in the central nervous system and spinal cord.

As used herein, the phrase “allele” or “allelic variant” has its ordinary meaning as understood in light of the specification and refers to a variant of a locus or gene. In some embodiments, a particular allele of a locus or gene is associated with a particular phenotype, for example, altered risk of developing a disease or condition, likelihood of progressing to a particular disease or condition stage, amenability to particular therapeutics, susceptibility to infection, immune function, etc.

As used herein, the term “amplification” has its ordinary meaning as understood in light of the specification and refers to any methods known in the art for copying a target nucleic acid, thereby increasing the number of copies of a selected nucleic acid sequence. Amplification may be exponential or linear. A target nucleic acid may be either DNA or RNA. Typically, the sequences amplified in this manner form an “amplicon.” Amplification may be accomplished with various methods including, but not limited to, the polymerase chain reaction (“PCR”), transcription-based amplification, isothermal amplification, rolling circle amplification, etc. Amplification may be performed with relatively similar amount of each primer of a primer pair to generate a double stranded amplicon. However, asymmetric PCR may be used to amplify predominantly or exclusively a single stranded product as is well known in the art (e.g., Poddar et al. Molec. And Cell. Probes 14:25-32 (2000)). This can be achieved using each pair of primers by reducing the concentration of one primer significantly relative to the other primer of the pair (e.g., 100-fold difference). Amplification by asymmetric PCR is generally linear. A skilled artisan will understand that different amplification methods may be used together.

As used herein, “amplicon” has its ordinary meaning as understood in light of the specification and refers to the nucleic acid sequence that will be amplified as well as the resulting nucleic acid polymer of an amplification reaction. An amplicon can be formed artificially, such as through polymerase chain reactions (PCR) or ligase chain reactions (LCR), or naturally through gene duplication.

The terms “individual”, “subject”, “host,” or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys), humans, cattle, horses, sheep, goats, swine, rabbits, dogs, cats, rodents, rats, mice, or guinea pigs.

As used herein, the term “liquid biopsy” has its ordinary meaning as understood in light of the specification and refers to the collection of a sample and the testing the sample, wherein the sample is non-solid biological tissue such as blood.

As used herein, the term “cfDNA” has its ordinary meaning as understood light of the specification, and refers to circulating cell free DNA, which includes DNA fragments released to the blood plasma. cfDNA can include circulating tumor deoxyribonucleic acid (ctDNA).

As used herein, the term “ctDNA” has its ordinary meaning as understood in light of the specification, and refers to circulating tumor DNA, which includes a tumor-derived fragmented DNA in the bloodstream that is not associated with cells.

### EXAMPLES

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### Example 1

cfDNA Extraction From Subjects

Embodiments of cfDNA isolation described herein was performed using a series of extractions. Blood samples were collected from canine subjects into anti-coagulant blood collection tubes (BCTs) containing cell free DNA stabilizing components. Non-limiting examples of viable collection tubes include the Roche Cell-Free DNA collection tubes, as well as Streck, Biomatrica, MagMax, or Norgen collection tubes. BCTs were then centrifuged to separate the plasma fraction and red blood cells. The cell free plasma layer was removed from the BCT and either stored or taken directly into cell free DNA (cfDNA) extraction.

cfDNA was extracted from 2-8 mL of plasma using a commercially available magnetic bead-based extraction kit (MagMax Cell-Free DNA Isolation Kit). Other comparable extraction methods/kits could potentially be used for this process, including column-based solid phase methods, and well as precipitation-based methods. cfDNA was eluted and quantified by fluorometry and electrophoresis (TapeStation).

Whole genome libraries were prepared from the cfDNA by contacting the cfDNA sample with random primers configured to amplify whole genomes for sequencing. However, it will be understood to those skilled in the art that any method suitable for sequence amplification could be utilized, such as next generation sequencing, for example. In one embodiment, library preparation may include incorporation of unique molecular identifiers and unique sample specific barcodes to allow for multiplexing of samples from different subjects.

### Example 2

### Fragmentomics Analysis Based Upon Fragment Size Distribution

Embodiments of analysis of the subject's cfDNA was performed through a comparison of the size, and distribution of cfDNA fragments in the plasma of canines compared to those in healthy canine subjects. Libraries were quantified to determine total concentrations and analyzed for fragment size by sequencing and analysis of the fragment lengths taking from the sequencing process.

Whole genome sequencing of libraries was accomplished by paired-end sequencing on a NovaSeq 6000 with 2×100 cycles of paired-end sequencing. However, it will be understood to those skilled in the art that many other cycles configurations like 2×50 or are suitable for paired-end sequencing and could be utilized. DNA fragment sizes were determined following a sequencing run by counting the number of nucleotides in each cfDNA fragment that was amplified in the library.

### Example 3

### Data Analysis to Positively Identify Subjects Having a Tumor or a Cancer

The following examples demonstrate performing fragment size distribution analysis on cfDNA fragments to determine whether a subject has a tumor or a cancer.

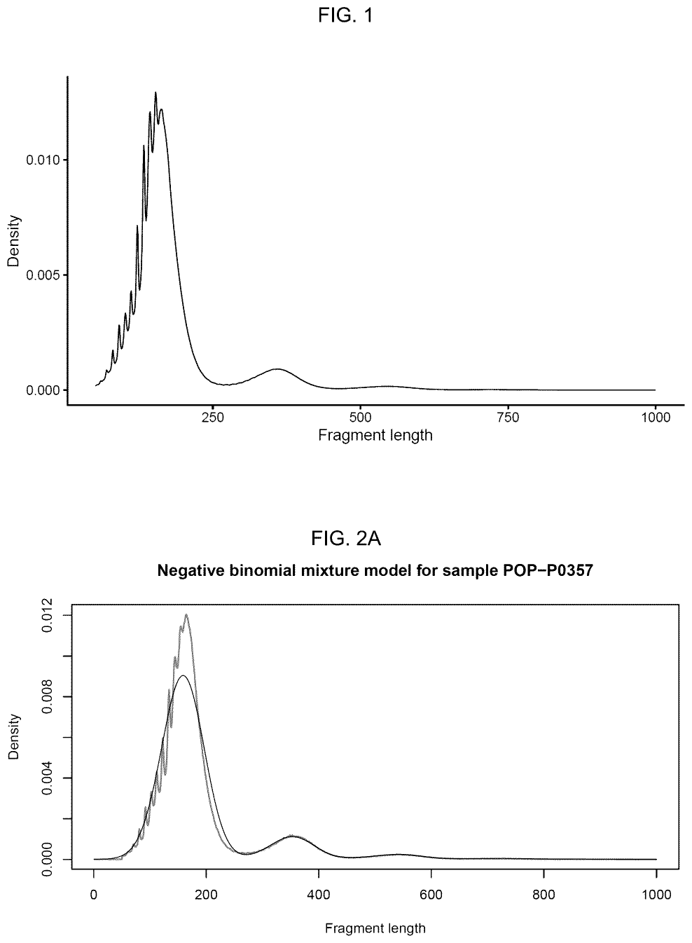

Twelve batches of samples comprising a mixture of both cancer, tumor, and normal cfDNA from a collection of canine subjects were obtained. The cfDNA was isolated, sequenced, and then analyzed to calculate the fragment size distributions based upon the sizes of the cfDNA fragments. In the series of 8 tests, batch IDs 1-7 and 12 were analyzed. These library batches ranged from approximately 2-5 million fragments. FIG. 1 depicts the distribution of fragment length in a batch of cfDNA samples taken from normal, healthy subjects.

The fragment size distributions across batches were measured directly or were utilized to form mixture models of each batch for comparison. As shown in FIG. 1, the distribution of fragment length is multimodal, with one mode per nucleosome, and visible oscillations on the shorter side of each nucleosomal peak. Therefore, a natural model choice is a mixture model.

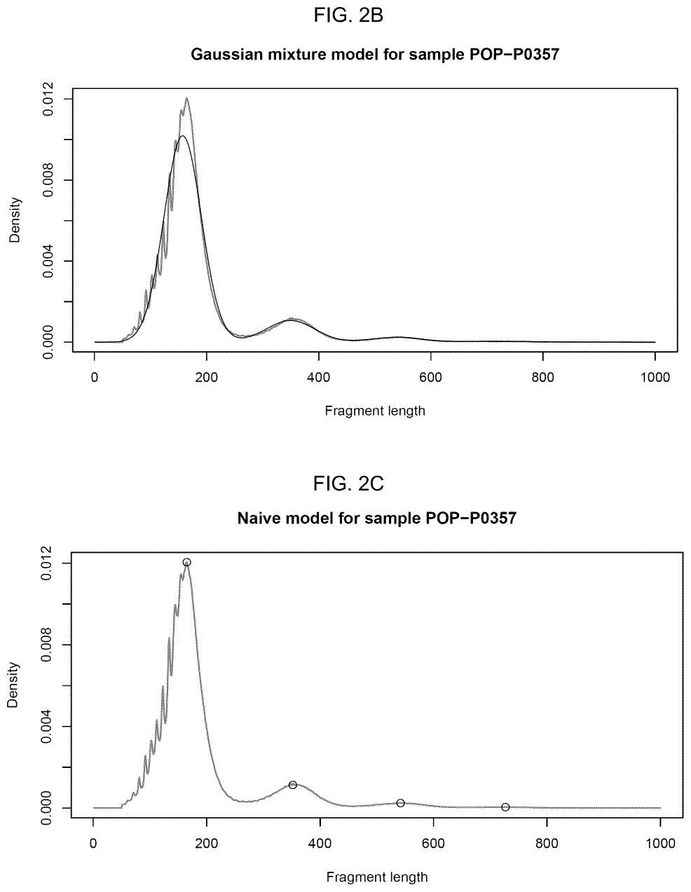

A mixture model, as described herein, has its ordinary meaning as understood by those of skill in the art, and refers to a probabilistic model for representation of subpopulations within a population, without a requirement that a given observed data set must identify the subpopulation to which an individual observation belongs. The counts of each fragment length are modelled with probability distributions. In one embodiment, these distributions are over dispersed Poisson distributions, also known as negative binomial distributions. Because these distributions are positively skewed, while the data have negative skew, the model is fitted on reversed data (length 1 becomes length 1000 and so on and vice versa) and the results are reversed again. An example of this is given in FIG. 2A, wherein a control sample (grey line) and its model fit (black line) are shown below for a 4-component negative binomial mixture. In a second embodiment, the data are modelled with a Gaussian mixture model (FIG. 2B). Under this model, the fragment size distributions are approximated with Gaussian distributions, which have the advantage of being symmetric about their mode. The Gaussian mixture better models the first peak, at the expense of the second one. In a third embodiment, the model consists essentially of smoothing the profile and identifying the locations of the peaks and their maximum heights (FIG. 2C).

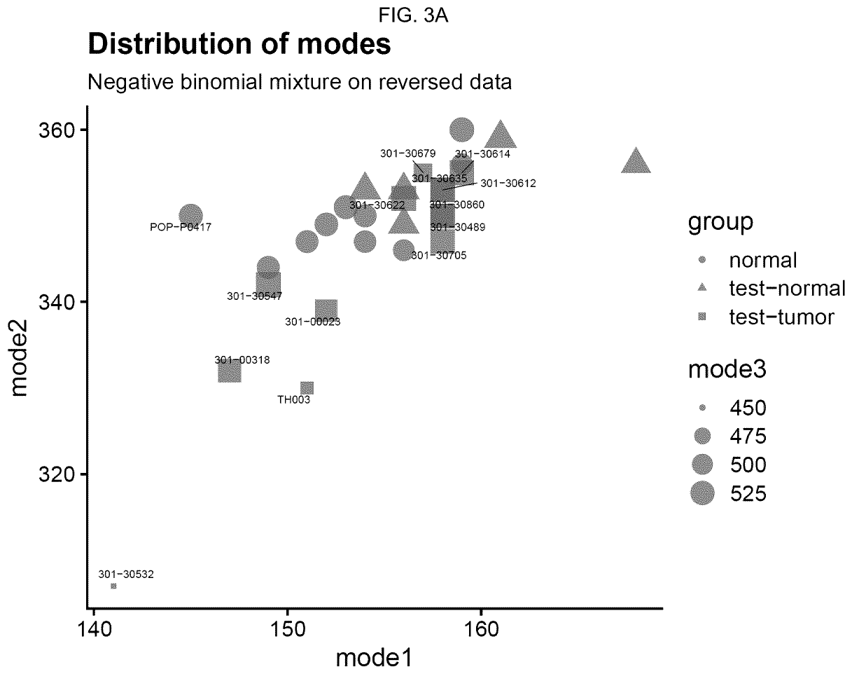

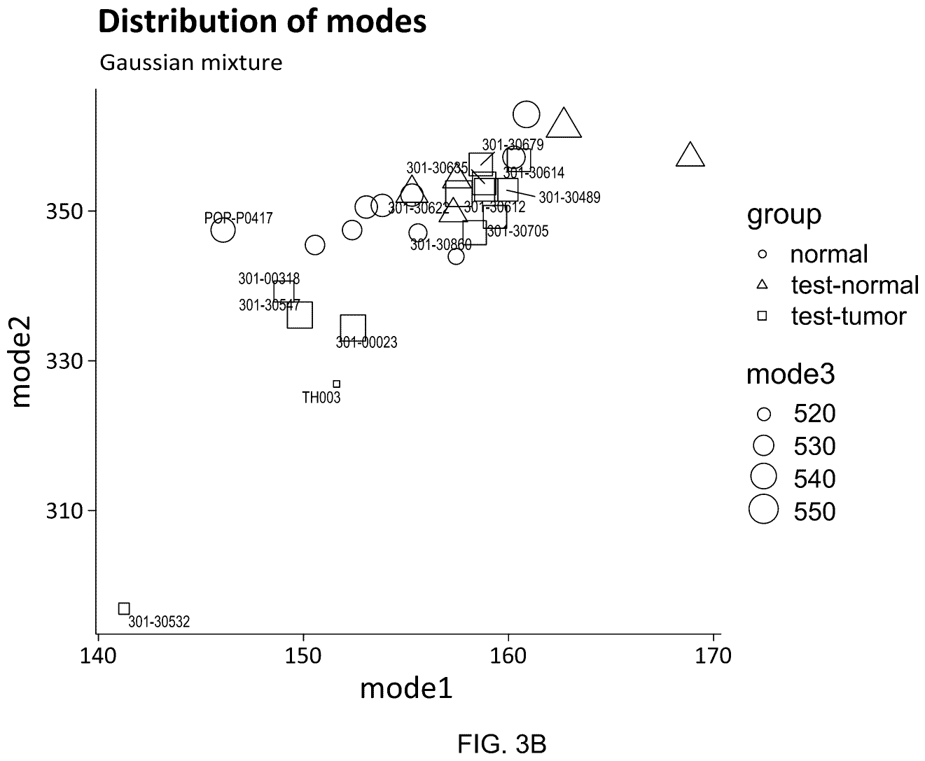

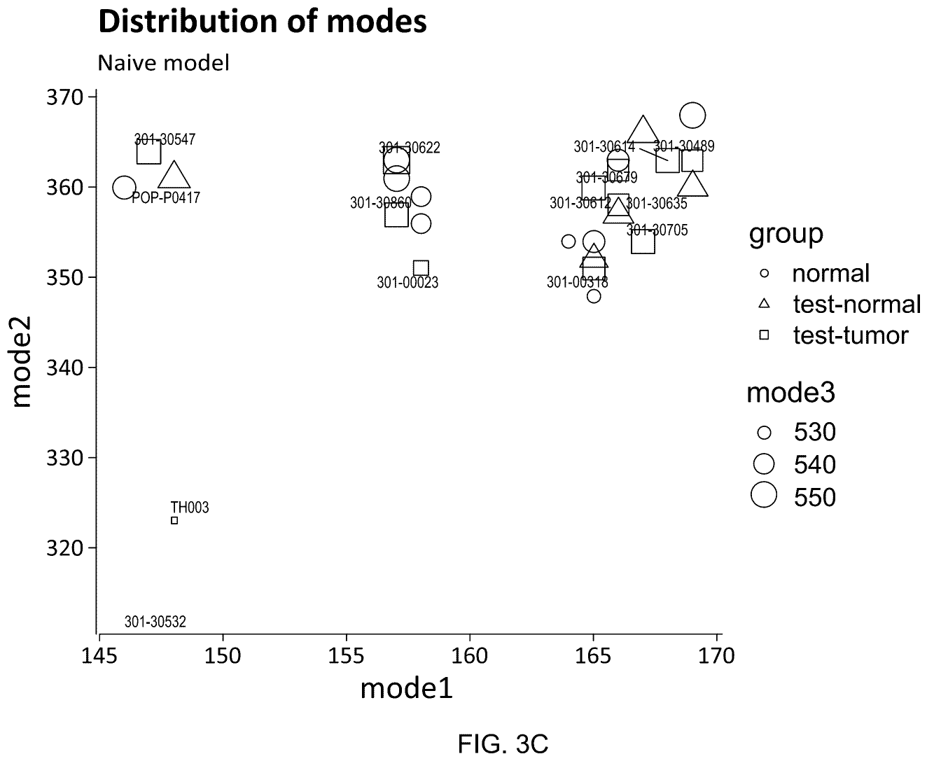

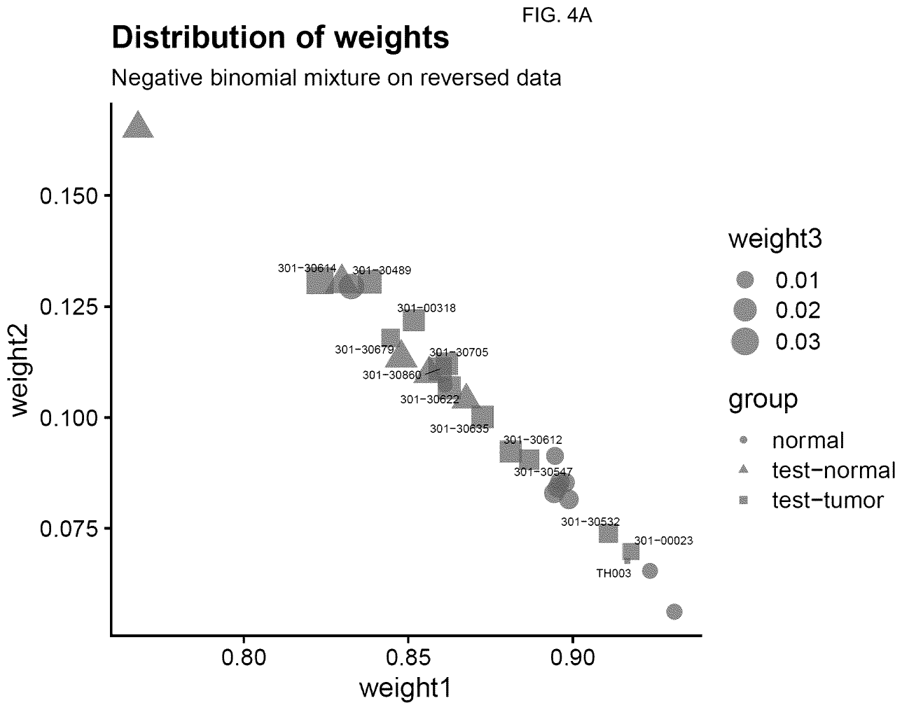

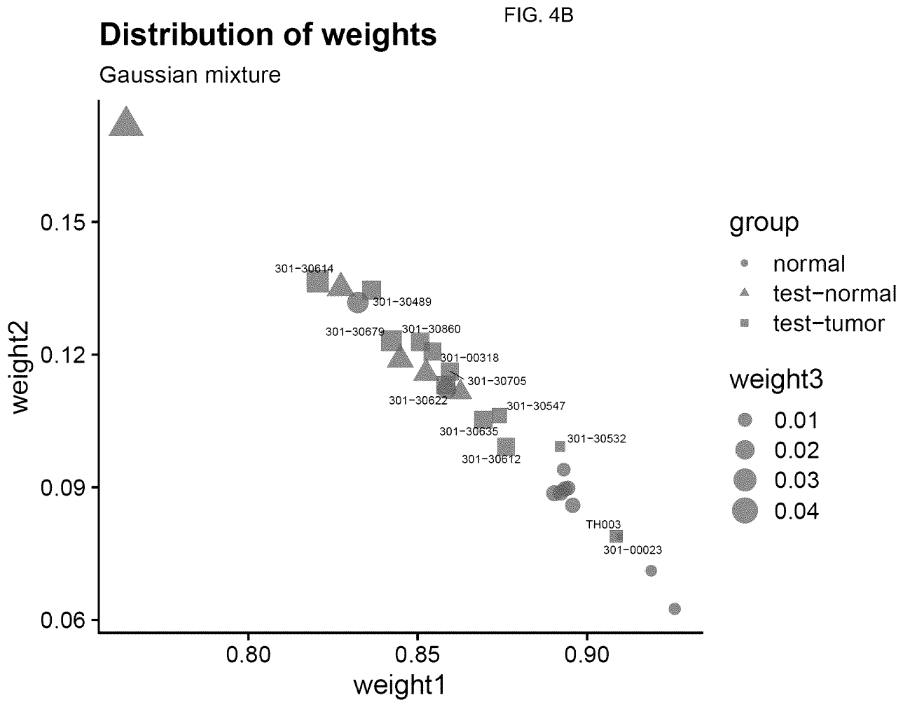

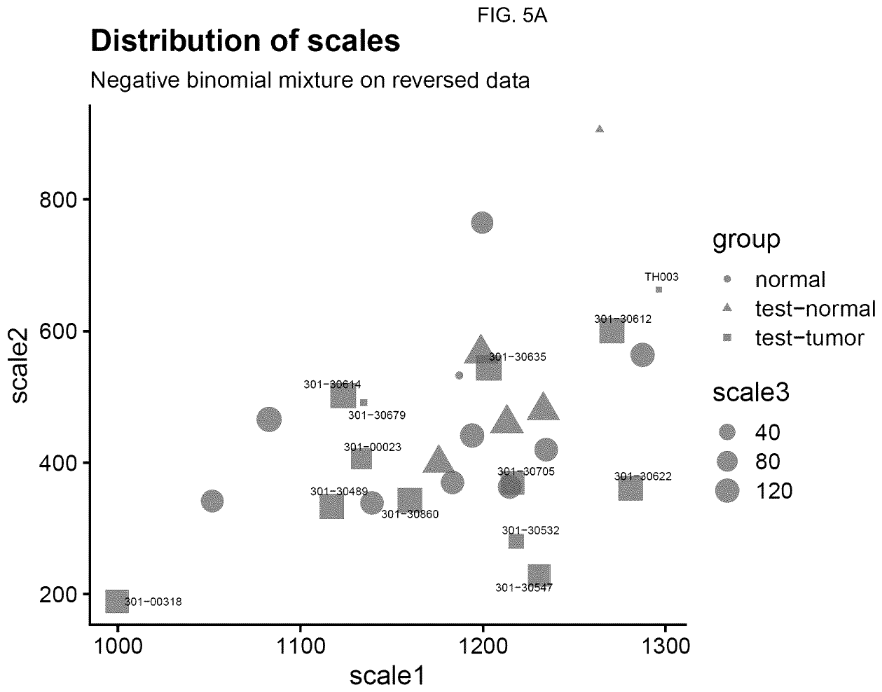

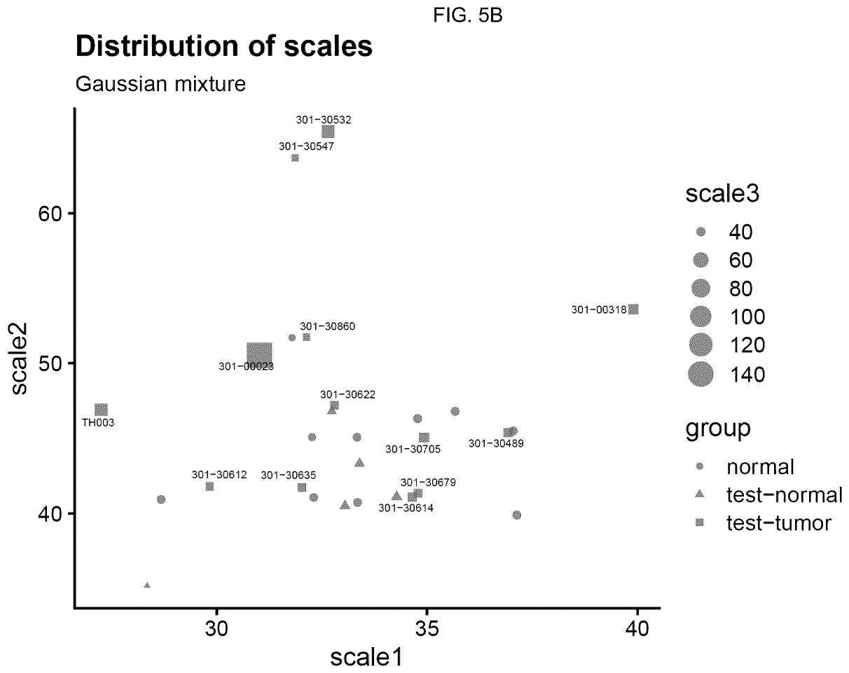

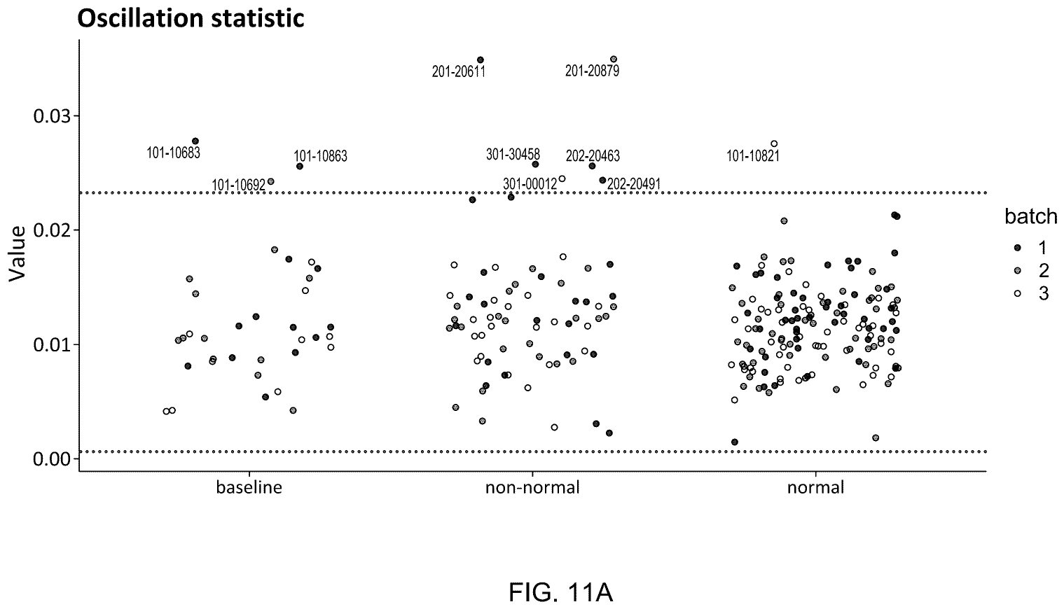

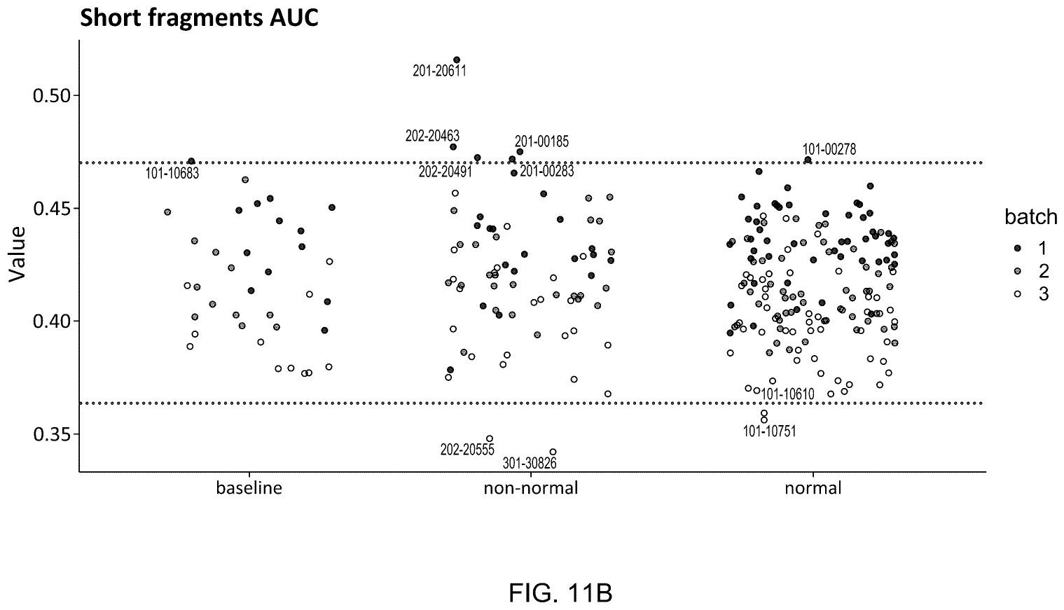

Despite the differences visible in FIGS. 2A-2C, the mixture models perform similarly with mode distribution (FIGS. 3A-3C). Normal samples are either those from the baseline run (circles), or ‘poppy’ samples from the TH run (here called ‘test’) (triangles). Labeled in FIGS. 3A-3C are the names of patient samples which were deemed normal. While all test samples that were called normal cluster with the control samples, some additional samples cluster there too. It is possible that from a fragmentomics perspective, these test samples are indeed normal. The distributions of weights inferred by the mixture models are shown in FIGS. 4A-4B, while the distributions of inferred scale parameters (overdispersion for the negative binomial mixture model and standard deviation for the Gaussian mixture model) are shown in FIGS. 5A-5B. Classification, whether by computing multivariate p-values or by machine learning methods, can consider mode locations, scale parameters, and weights, either independently or jointly. Additional features may include a measure of the amplitude of the oscillations, the area under the curve (AUC) of the fragment size profile for short fragments (FIGS. 11A-11B), and other length intervals. The correlation of these features is shown in FIG. 15.

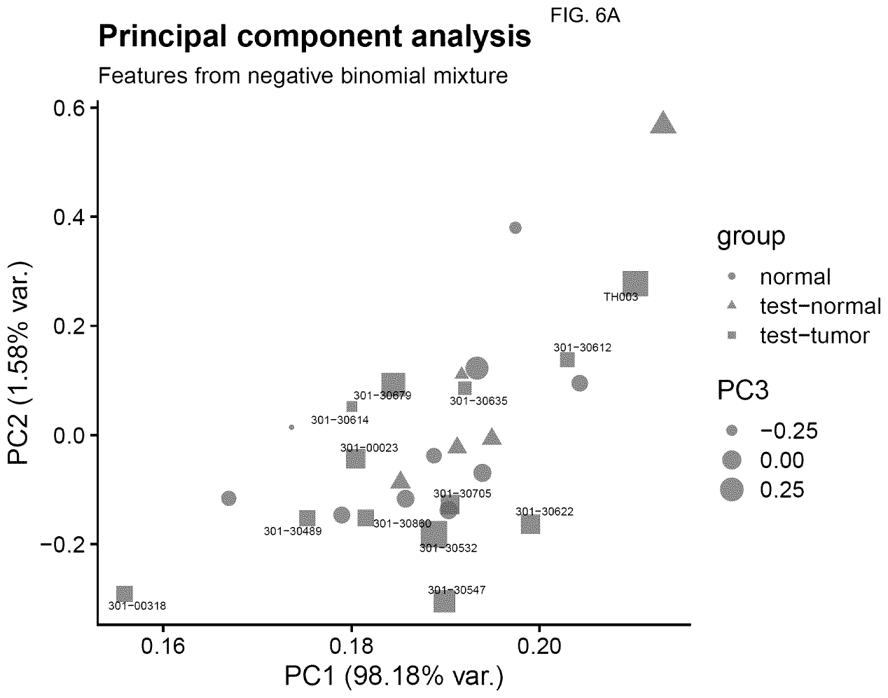

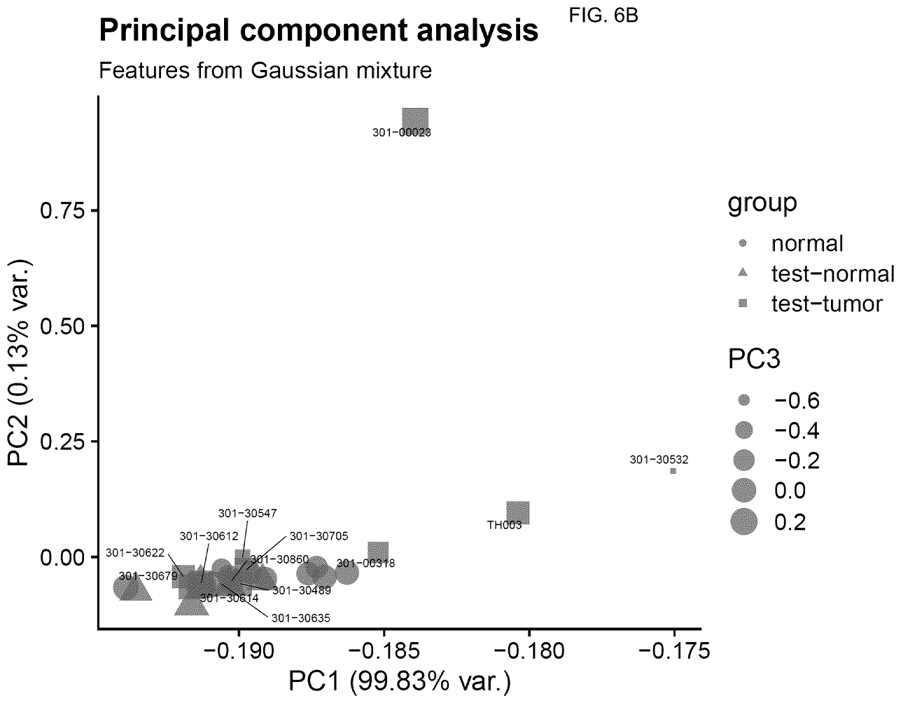

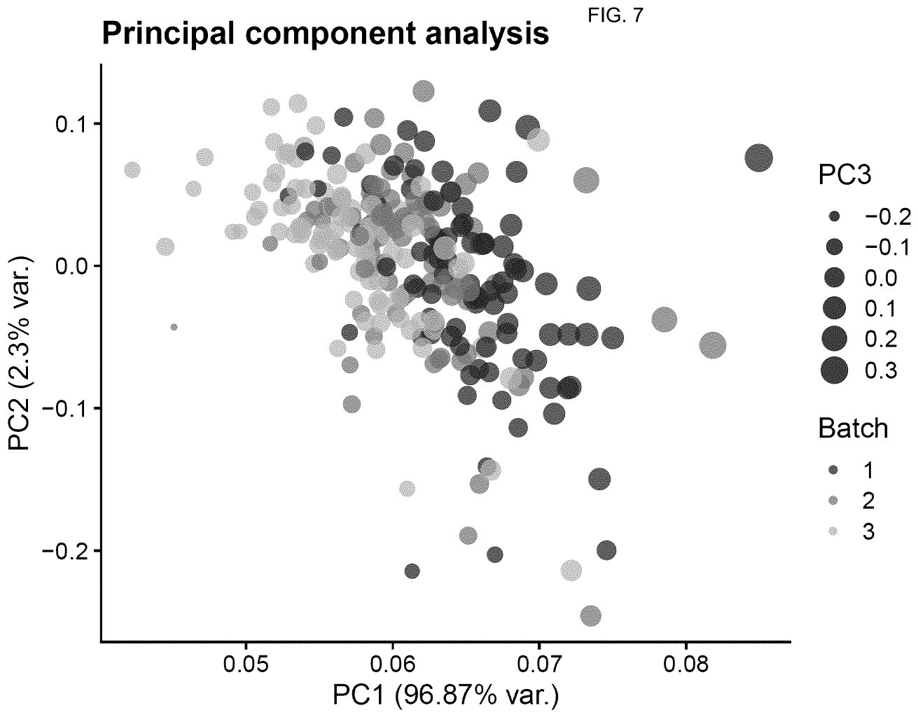

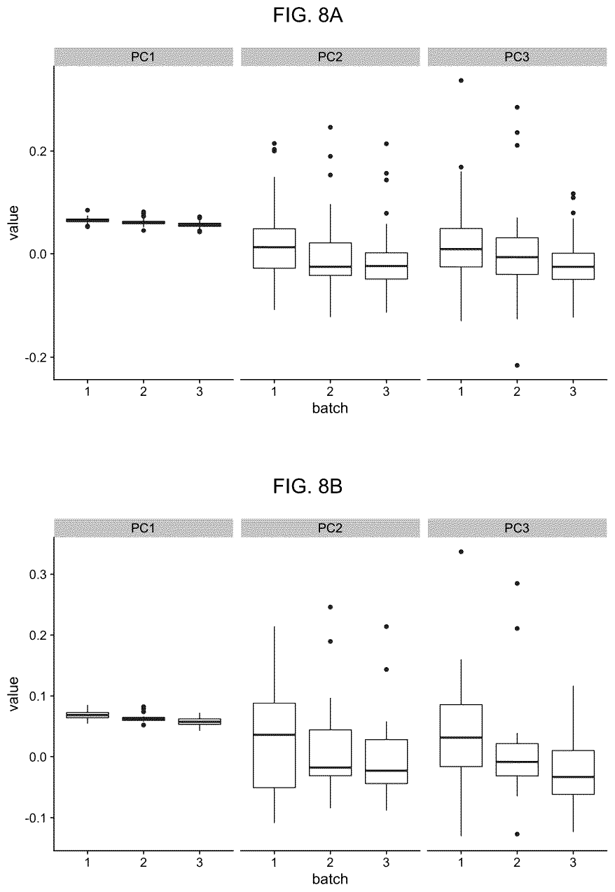

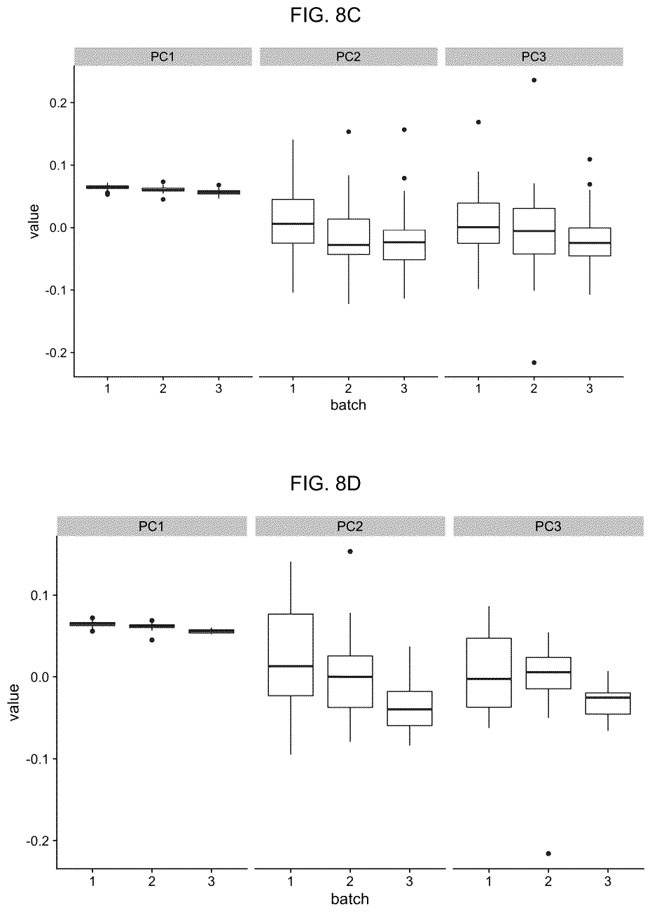

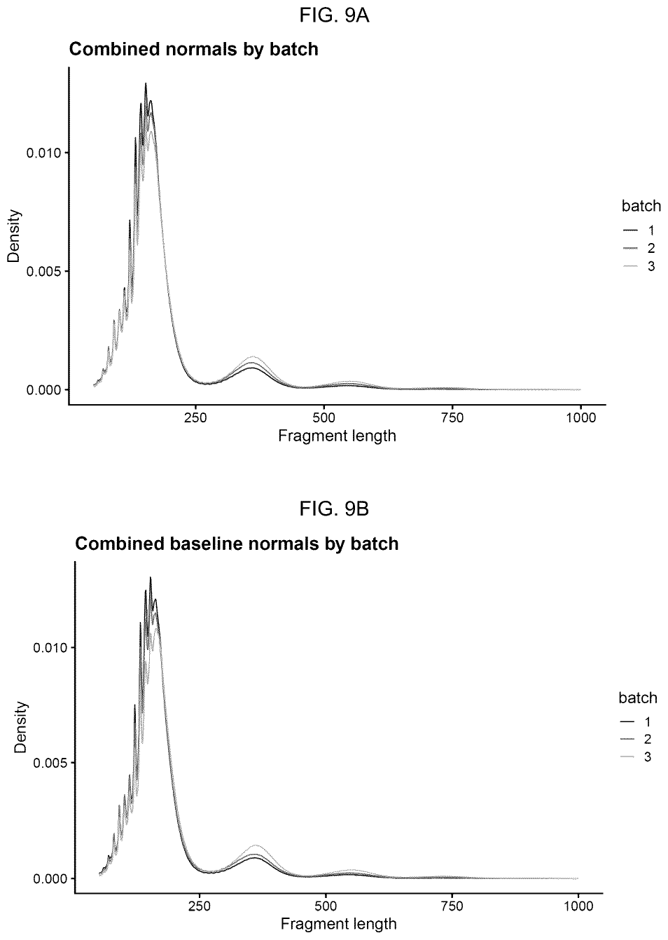

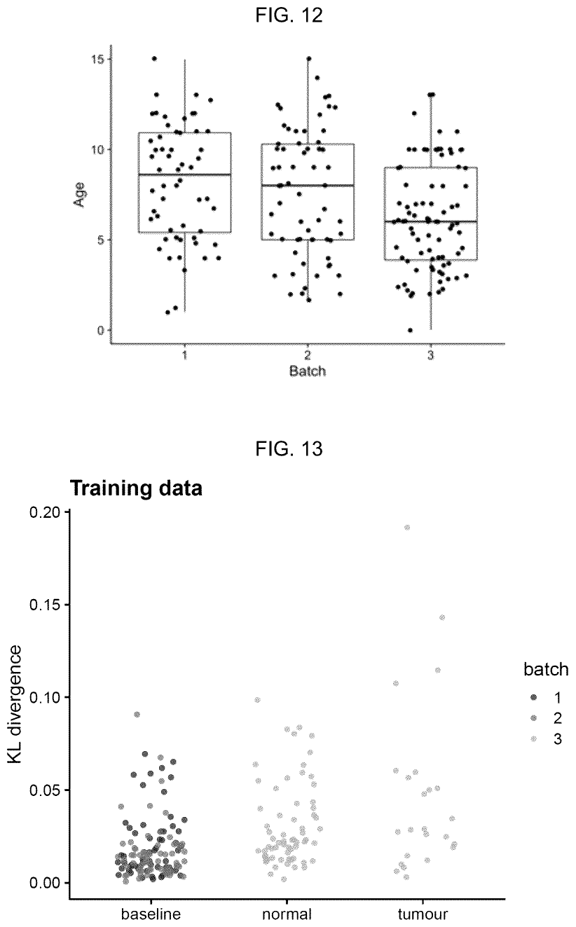

As disclosed herein, the values of the extracted features are affected by the batch (FIGS. 6A-6B and 13-14). Specifically, as the batch number increases, samples generally move to the top-left corner of the PCA analysis shown in FIG. 7. This is also visible in the boxplot analysis of calculated PC values by batch across PC1-3, which capture 99.65% of the data variance (FIGS. 8A-8D). In practice, the trend serves to amplify the higher nucleosome peaks in larger batches (FIGS. 9A-9B). As disclosed herein, the baseline set is slightly skewed to older subjects (FIG. 12). It is subsequently envisioned that in some embodiments, analysis may comprise correlating the fragment size profile with age.

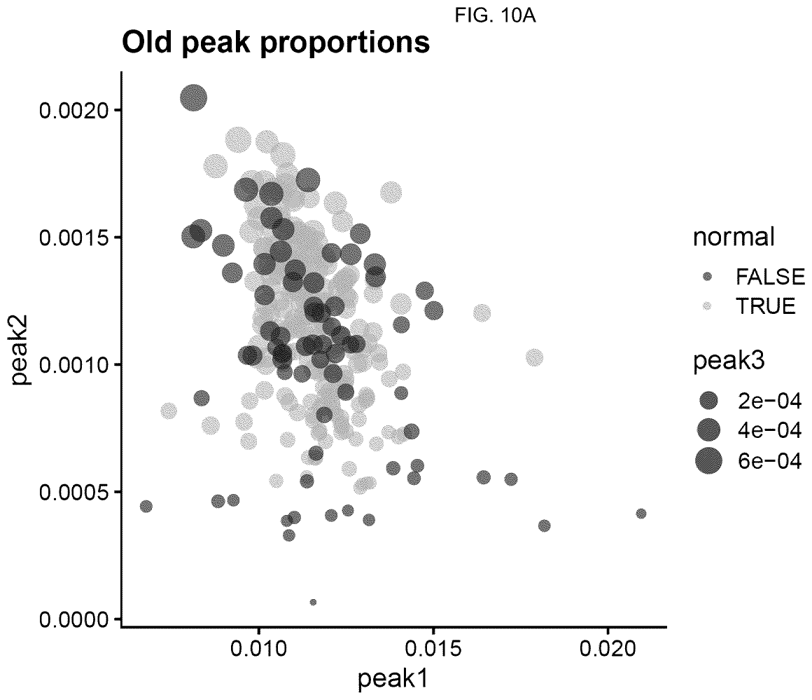

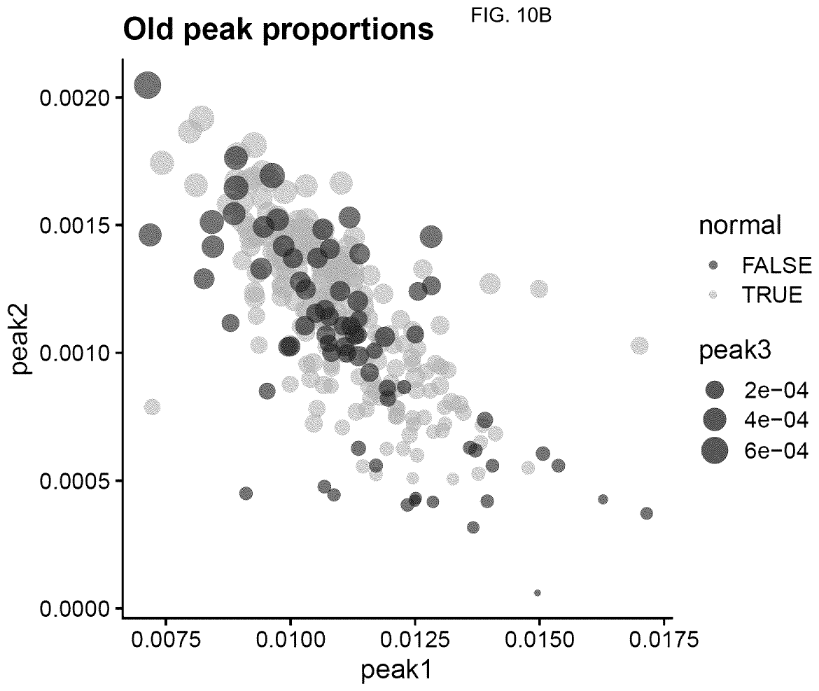

The initial set of statistics used to describe and classify samples revolved around a reference normal sample, consisting of the combination of one or more normal samples. Peak locations were identified in the smoothing of this profile, and peak proportions were calculated at these locations in the normalized profiles. Additionally, the KL divergence from the combined normal samples was computed. Under these statistics, there were observable batch effects (FIG. 9A). Secondly, the absolute difference of the proportions of all peaks between sample and the combined normals was used. Under this statistical analysis, normal samples have lower values, meaning that their peak proportions are more similar to the combined normals, which in turn means that samples containing tumor material have altered proportions of the observable nucleosomal peaks (FIGS. 10A-10B). Finally, the KL divergence computes a distance between two probability distributions, in this case a sample's fragment size distribution in the range 51-1000 bp and the distribution in the reference normal sample.

As disclosed herein, normal samples have a smaller KL divergence from the reference normal sample, and this holds true whether the reference normal sample is made up of all normals or only the baseline samples. Nevertheless, there is a large overlap in the two distributions. Alternative statistics are obtained from a Gaussian mixture model that is fit to the fragment size distribution in the range 51-1000 bp. The mixture has four components, one of each observable nucleosomal peak. The parameters (4 means, 4 standard deviations, 3 mixture weights as the fourth is obtained by 1—sum of the first three weights) are learnt for each sample by Markov chain Monte Carlo (MCMC).

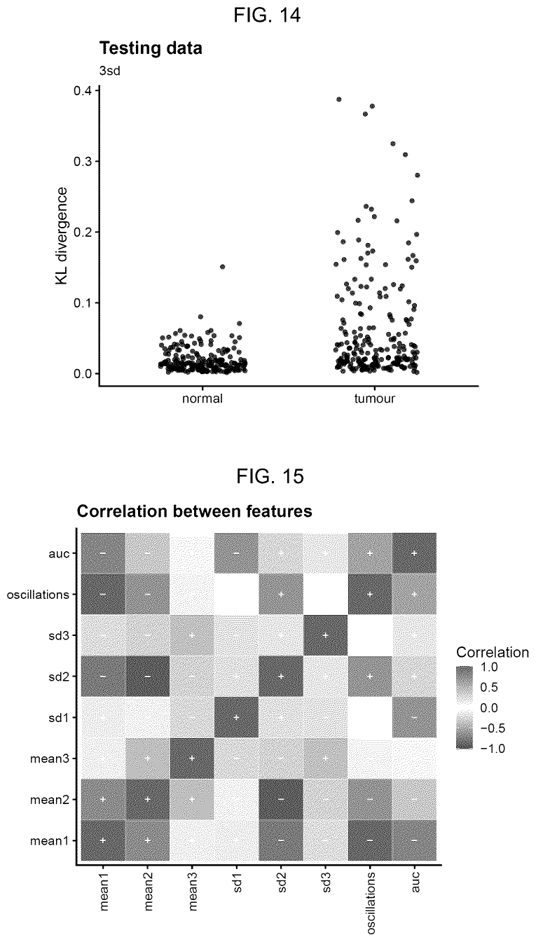

A subset of known, normal fragment size distributions was utilized to form the baseline set, from which the reference was computed. The KL divergence values were different across baseline, normal, and tumor samples, with the highest values in tumor samples, and the lowest in baseline (FIGS. 13-14). Four thresholds were considered in comparing the KL values of experimental samples to the KL values of normal samples: (1) the maximum value observed in the normal group (“max”), (2) the mean of the two largest values observed in the normal group (“mean”), (3) three standard deviations about the mean KL in the normal group (“3 sd”), and (4) four standard deviations about the mean KL in the normal group (“4 sd”).

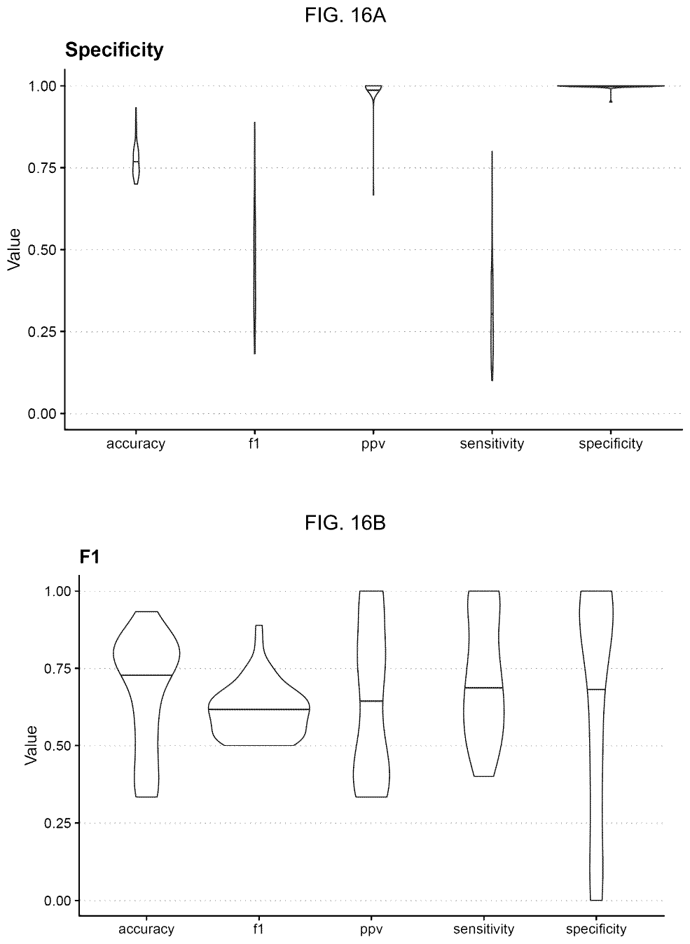

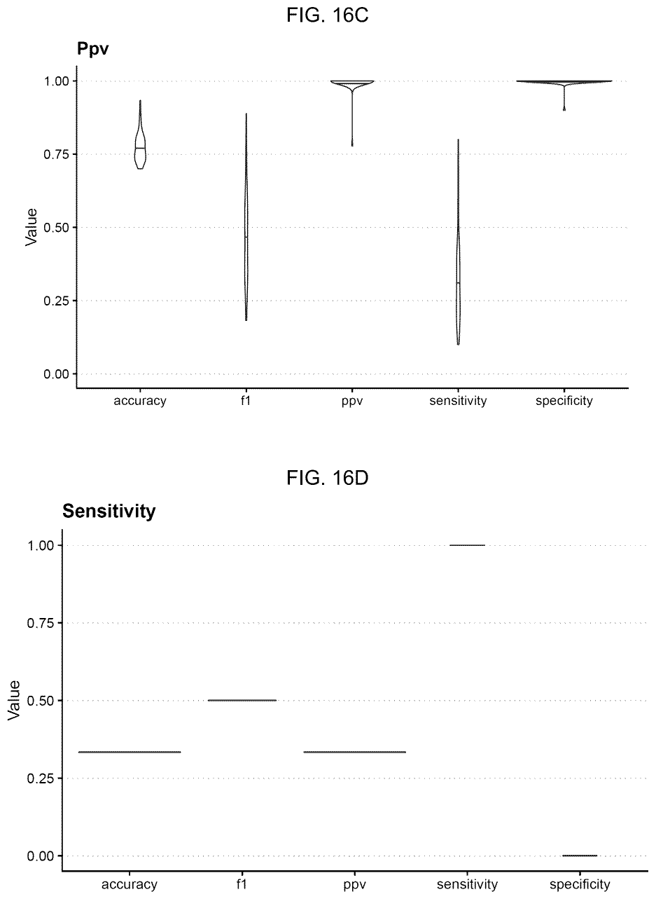

A threshold may be selected to optimize a specific criterion. For example, accuracy, sensitivity, specificity, PPV, and the F1-score were computed for each threshold using data from Batches 1-3 (Table 1). Table 2 provides the performance metrics for Batches 4-7 and 12. Optimization of each of these metrics produced a different result (FIGS. 16A-16D). As disclosed herein, optimization of sensitivity and specificity was not a good objective since pathological solutions are preferred. Prioritizing specificity, the outcomes that optimize PPV appear to be a good compromise. Classifying samples as tumor or normal is a discriminative learning task that can be approached in other ways that typically do not depend on a baseline set composed only of normals, but rather on training data using both labels. Non-limiting examples of such approaches include:
a) Logistic regression (LR) regularized with a penalty such as ridge, lasso, grouped lasso, fused lasso, or others.
b) Support-vector machines (SVM).
c) Neural networks (NN), with one or more hidden layers.

These classification approaches could use as features the normalized counts in a chosen range (e.g. 51-1000 bp), or features extracted from the data, as described above in the context of the mixture models.

TABLE 1
Performance Metrics for Batches 1-3

| Statistic | Max | Mean |
|---|---|---|
| True Positives | 4 | 4 |
| False Positives | 0 | 1 |
| True Negatives | 65 | 64 |
| False Negatives | 20 | 20 |
| Accuracy | 0.7753 | 0.764 |
| Sensitivity | 0.1667 | 0.1667 |
| Specificity | 1.0 | 0.9846 |
| Positive Predictive Value | 1.0 | 0..8 |
| F1 Score | 0.2857 | 0.2759 |

TABLE 2
Performance Metrics for Batches 4-7 and 12

| Statistic | 3SD |
|---|---|
| True Positives | 47 |
| False Positives | 1 |
| True Negatives | 190 |
| False Negatives | 170 |
| Accuracy | 0.5809 |
| Sensitivity | 0.2166 |
| Specificity | 0.9948 |
| Positive Predictive Value | 0.9792 |
| F1 Score | 0.3547 |

Analysis of Batches 4-7 and 12 identified 47 true positive results (i.e. 47 samples were correctly identified as being a tumor or a cancer), and 4 true positive results from Batches 1-3.

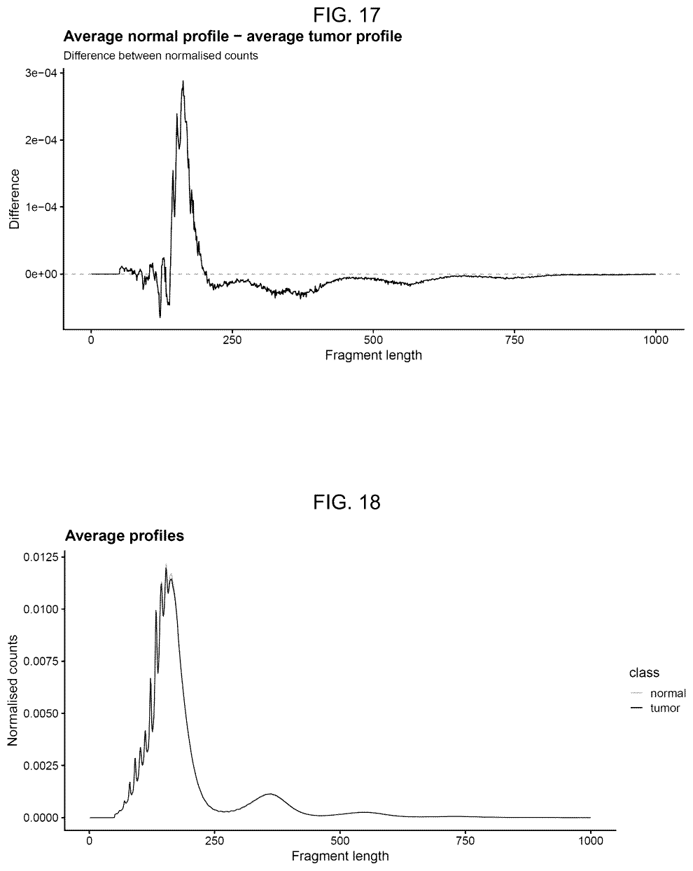

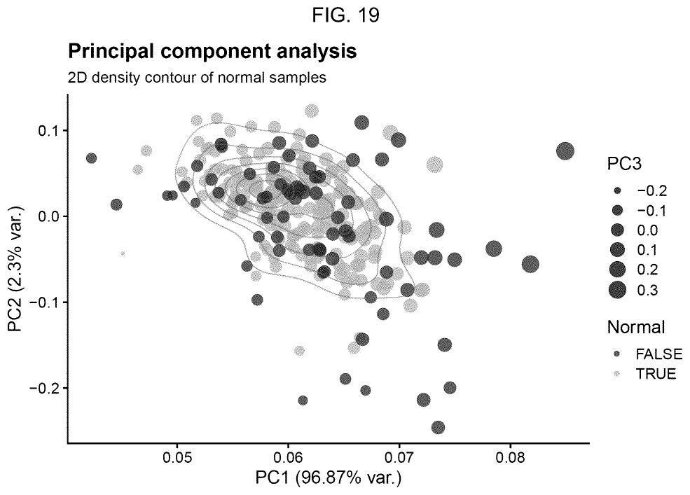

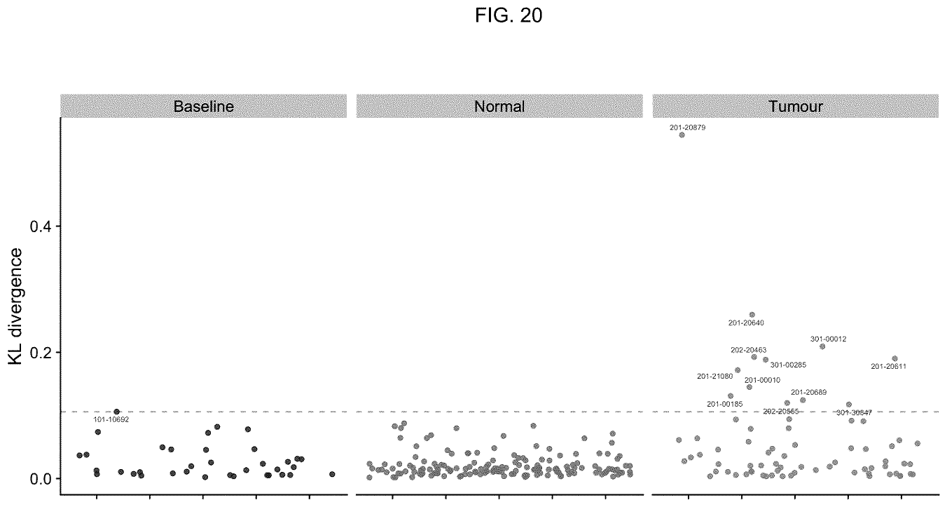

Crucial for classification is a distinction between the data distributions in the two classes. When taking the average profiles per class and subtracting one from the other, there was an excess of fragments around ˜150 bp in the normal samples, and an excess of the multiple peaks of longer fragments in the tumor samples (FIG. 17). The differences, however, are small, and barely visible when the two average profiles are plotted against each other (FIG. 18). A PCA of all samples shows significant overlap between the normal and tumor groups (FIG. 19). This is probably because genuine tumor samples may have such low tumor content that the fragment size profile is effectively normal. The normal samples form a tight cluster, suggesting that their profiles are robustly reproducible. The tumor samples, however, can differ from the normals in different ways, and this complicates the description of a distribution for their class. Therefore, outlier detection may be preferable to classification with, for example, logistic regression. Based on the observations that (i) all normals are similar to each other, (ii) tumor samples may differ in different ways, and (iii) wanting to use the whole data without modelling approximations, another outlier detection approach uses a distance function, for example the KL divergence, between a test sample and the average of the baseline samples (FIG. 20). FIG. 20 shows the baseline sample with the largest KL divergence and all tumor samples above this threshold.

Typically, cfDNA fragment size analysis to detect cancer has previously been designed based on human data. It was surprisingly discovered using the techniques described herein that a distinguishing element of the samples (companion animal samples) is that there are more peaks in the fragment size profiles in companion animal samples than human. By taking into consideration of the entire fragment size profiles, the methods described herein indirectly benefit from the presence of these additional peaks. Thus, the presence of the multiple peaks is advantageous over prior methods, and previously unknown.

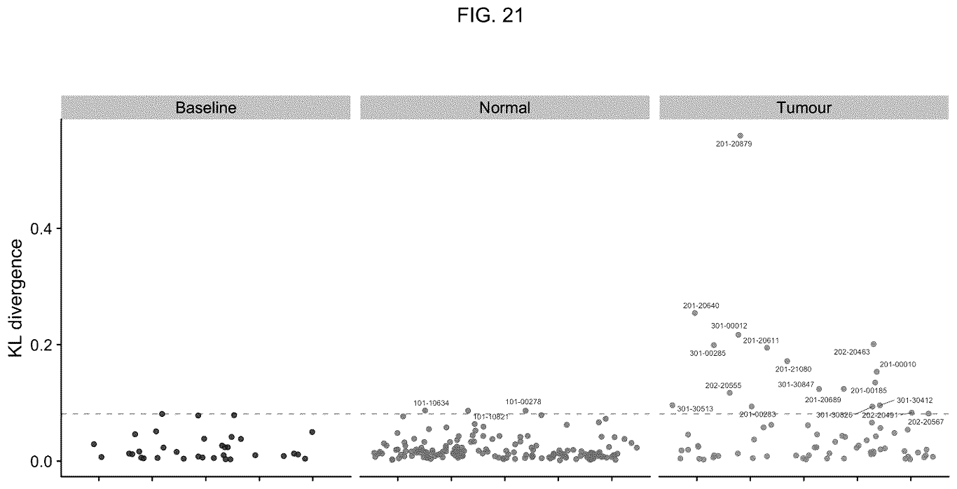

Removing outlier samples from the baseline resulted in more samples significantly different from normal for KL divergence, but also some false positives (FIG. 21). Testing datasets with the analysis methodology disclosed herein yields slightly better results than the probabilistic approach above, in terms of higher specificity as well as higher sensitivity.

### Example 4

### Fragmentomics-Based Tumor Content Estimation

The following example demonstrates a summary of the methodology for performing fragmentomics-based tumor content estimation.

1. The probabilistic model:

t˜Beta(1, 10)

θ_{N}˜Dirichlet(α_{N})

θ_{T}˜Dirichlet(α_{T})

Q=|θ_{N}θ_{T}|W(t)

y.˜Multinomial(q.)

The probabilistic model above defines three unknown parameters with their priors, a deterministic calculation, and finally the likelihood model. The observed data was stored in a matrix Y, with one column per copy number (CN) (e.g. 1, 2, 3).

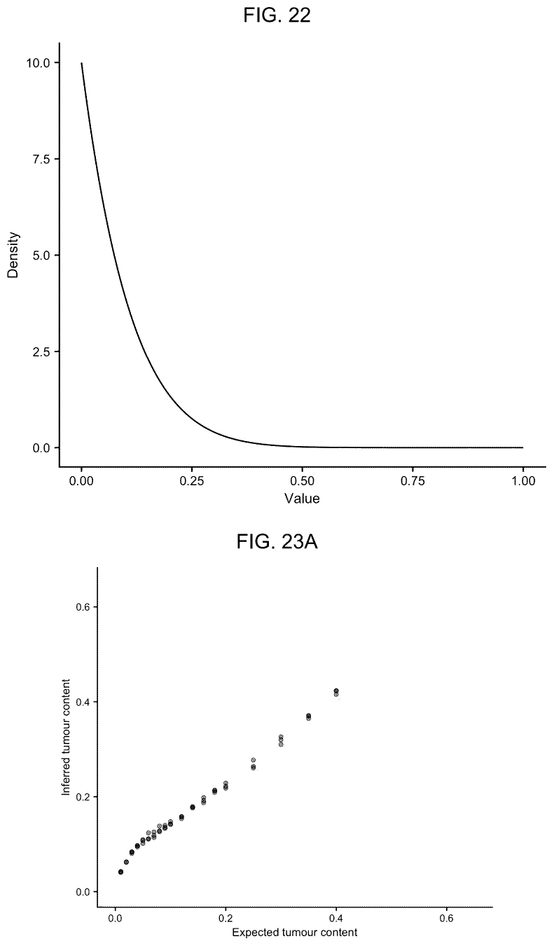

The first unknown is the tumor content (TC) t, which was given a prior favoring small values. The prior had no information about the sample under consideration. A monotonically decreasing curve was used to avoid exploring the alternative solution that each of these deconvolutions admits: swapping the normal and tumor labels of the parameters theta. The prior distribution is depicted in FIG. 22.

thetaN and thetaT are the pure normal and tumor profiles. Their Dirichlet prior ensures non-negativity and unit-sum. The parameters of the prior distributions were obtained from model 5's estimates of the profiles, with deconvolutions based on the following equations. As such, these priors were empirical, based on some aspects of the data.

2. Model 5's equations:

$$\\ \begin{matrix}{θ}_{N} = \frac{3 ( 2 - t )}{4 ( 1 - t )} {\overline{y}}_{L} - \frac{2 + t}{4 ( 1 - t )} {\overline{y}}_{G} \\ = \frac{3}{1 - t} {\overline{y}}_{N} - \frac{2 + t}{1 - t} {\overline{y}}_{G} \\ = \frac{2 - t}{1 - t} {\overline{y}}_{L} - \frac{1}{1 - t} {\overline{y}}_{N}\end{matrix}$$

$$\\ \begin{matrix}{θ}_{T} = \frac{2 + t}{2 t} {\overline{y}}_{G} - \frac{2 - t}{2 t} {\overline{y}}_{L} \\ = \frac{2 + t}{t} {\overline{y}}_{G} - \frac{2}{t} {\overline{y}}_{N} \\ = \frac{2}{t} {\overline{y}}_{N} - \frac{2 - t}{t} {\overline{y}}_{L}\end{matrix}$$

Here, Ybar denotes the normalized count data. For example, YbarG is the gain profile (CN 3) divided by the total number of reads in the gain profile:

Ybar[,“gain”]=Y[,“gain”]/sum(Y[,“gain”]). (1)

Because the equations depend on the unknown TC t, they were computed for each value oft between 1 and 99% in increments of 1%. Given t, the equations were solved and estimates obtained for the pure profiles. With all these values, estimates of the data were created (see Q in the model above) and compared with the observed data. Model 5 used a normal distribution and selected the value of t (and consequently the pure profiles it generates given the data) that produced the highest log-likelihood (best fit). The solutions of the above equations may contain negative values. These were replaced by 0 before the estimates of the data are produced. Solutions with more than 20% non-positive entries were ignored; typically these occurred around extreme values of TC for samples with intermediate TC.

The parameters alpha were rescaled and biased versions of the model 5 estimates:

alpha_n=theta_n/sum(theta_n)*scaling_factor+bias. (1)

The scaling factor was 6M, where M is the length of the fragmentomics profile (number of rows of the Y count matrix); the bias was 1.

The final row of the model shown at the top is the multinomial likelihood.

For computational efficiency, instead of analyzing all M positions (51-260, M=210) of the fragment length profiles, the data was halved by considering only every second position (51-259 in increments of 2, M=105).

The model was implemented using the software Stan and run for 12,000 warm-up iterations, followed by 3,000 sampling iterations. Four chains were run in parallel, with parameters initialized as follows: (1) t is set to 5%, (2) thetaN was sampled from its prior (Dirichlet(alphaN)), and (3) thetaT was sampled from its prior (Dirichlet(alphaT)).

One control parameter was set: max_treedepth=20.

The model was developed using two sets of in silico mixtures, and tested on two additional sets of mixtures. Each dilution was produced and analyzed in triplicates to assess the model's robustness to resampling the data and converging to the solution. In one case out of 225 (57*3+54), the model failed to converge. There were 19 dilution levels, each created in triplicate=57 samples; three sets of mixtures produced 19*3=57 samples, while one produced 18*3=54 samples because its unmixed TC was less than our largest dilution level.

The initial mixtures consisted of samples 201-20885 and 201-00316 mixed into healthy cfDNA. The normal sample was chosen so as to have a fragmentomics profile as similar as possible to the pure normal signal found in the cancer samples.

The KL divergence between the pure normal signal in 201-20885 and its “matched normal” was around 0.003, while for 201-00316 it was 0.03. This larger value makes 201-00316 a more difficult mixture to analyze because it violates the assumption that there are only 2 signals in the data (normal and cancer). Instead, two normals and cancer were obtained, all at different proportions.

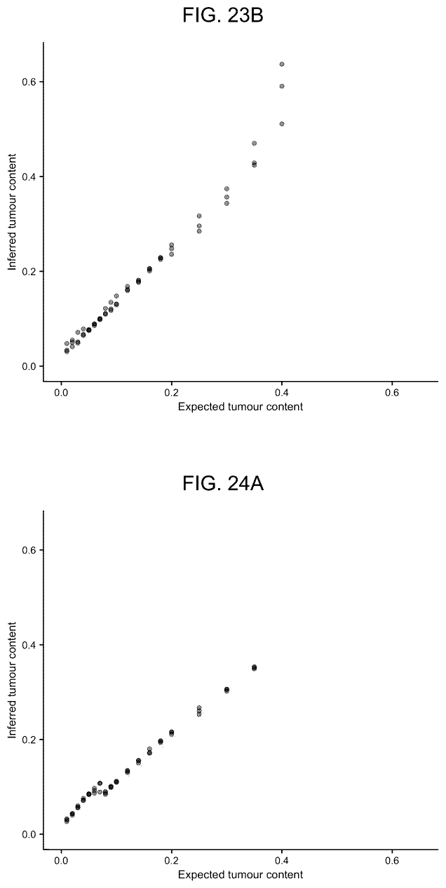

In both cases the TC was slightly overestimated (FIGS. 23A and 23B). This effect was more pronounced at lower TCs for 201-20885 but higher TCs for 201-00316. The expected TC was obtained from the original TC estimate of the undiluted sample.

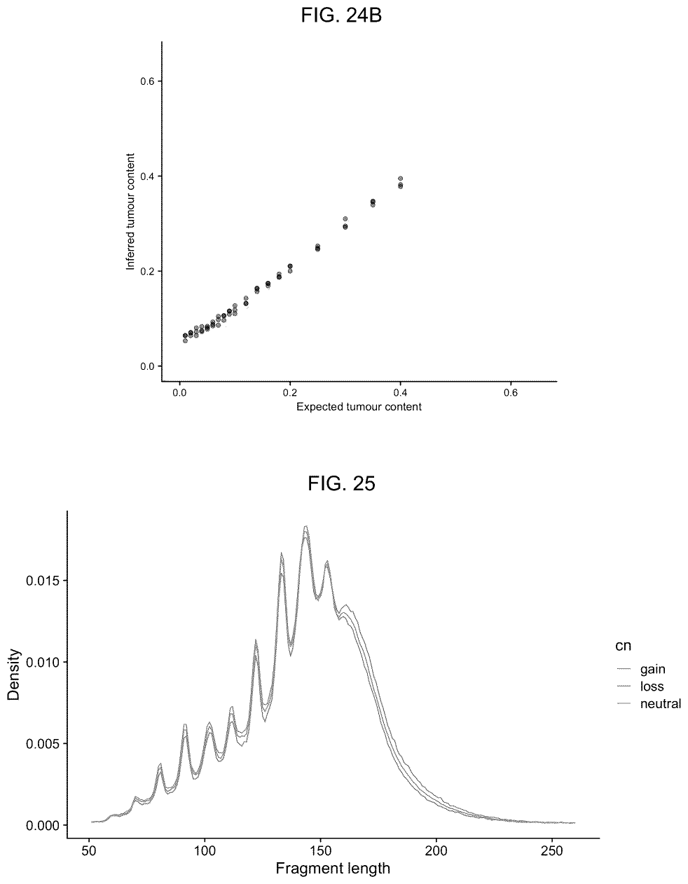

Because the model relied on the above mixtures for development, two more sets of mixtures were created to test the performance (FIGS. 24A and 24B). Closely matching normal samples for both of these cancer samples were obtained (KL divergence˜0.003). The TC was overestimated, but this was limited to small TC values (<10%).

### Example 5

### In Silico Mixtures

The following example demonstrates a summary of the approach for creating in silico mixtures of tumor and normal samples. In order to have a ground truth when benchmarking the estimation of tumor content, in silico mixtures of tumor and normal samples were created, so as to know and control the mixing proportions of the pure profiles.

cfDNA samples with high tumor content were mixed into a healthy cfDNA sample. To create standards for fragmentomics, the fragment length profile of the healthy cfDNA sample must match the fragment length profile of the normal component of the cancer-containing cfDNA sample.

Samples were screened to identify those with clean signal and high tumor content. 201-20885 displayed was good separation of the CN-specific fragment length profiles, and its TC estimate was 51% ([45%-56.8%]), in agreement with ichorCNA.

Sample 201-00316 had limited regions at CNs 1 and 3, but we observed what appear to be CNs 4 and 5. As expected, gains at CNs 4 and 5 appeared even more biased towards short fragments (FIG. 25). We estimated a TC of 43.7% ([33.5%-58.1%]) based on CNs 1, 2, and 3, less than predicted by ichorCNA.

The normal sample chosen to represent the normal component of 201-20885 was 101-10849 (KL 0.003 from the pure profile); for 201-00316 there was 101-00013 (KL 0.036 from the pure profile).

Selected samples 201-20885 and 201-00316 had approximately 51% and 44% tumor content, respectively. Note that, because 201-00316 had lower tumor content and a normal sample that was slightly different from its normal signal and with fewer total reads, the mixtures of this sample represented more difficult scenarios for the deconvolution. Mixtures of each of these two cancer samples were created in triplicates at the proportions shown in Table 3.

TABLE 3

| TC in | Proportions | | Proportions | |
|---|---|---|---|---|
| mixture | 101-10849 | 201-20885 | 101-00013 | 201-00316 |
| 1% | 0.98041681 | 0.01958319 | 0.97711634 | 0.02288366 |
| 2% | 0.96083362 | 0.03916638 | 0.95423269 | 0.04576731 |
| 3% | 0.94125043 | 0.05874957 | 0.93134903 | 0.06865097 |
| 4% | 0.92166724 | 0.07833276 | 0.90846538 | 0.09153462 |
| 5% | 0.90208405 | 0.09791595 | 0.8855817 | 0.1144183 |
| 6% | 0.8825009 | 0.1174991 | 0.8626981 | 0.1373019 |
| 7% | 0.8629177 | 0.1370823 | 0.8398144 | 0.1601856 |
| 8% | 0.8433345 | 0.1566655 | 0.8169308 | 0.1830692 |
| 9% | 0.8237513 | 0.1762487 | 0.7940471 | 0.2059529 |
| 10% | 0.8041681 | 0.1958319 | 0.7711634 | 0.2288366 |
| 12% | 0.7650017 | 0.2349983 | 0.7253961 | 0.2746039 |
| 14% | 0.7258353 | 0.2741647 | 0.6796288 | 0.3203712 |
| 16% | 0.686669 | 0.313331 | 0.6338615 | 0.3661385 |
| 18% | 0.6475026 | 0.3524974 | 0.5880942 | 0.4119058 |
| 20% | 0.6083362 | 0.3916638 | 0.5423269 | 0.4576731 |
| 25% | 0.5104202 | 0.4895798 | 0.4279086 | 0.5720914 |
| 30% | 0.4125043 | 0.5874957 | 0.3134903 | 0.6865097 |
| 35% | 0.3145883 | 0.6854117 | 0.199072 | 0.800928 |
| 40% | 0.2166724 | 0.7833276 | 0.0846538 | 0.9153462 |

### Example 6

### Quantifying the Separation Between CN-Specific Fragment Length Curves

This example outlines methodology for quantifying the separation between fragment length curves in sample analysis.

Fragment length profiles can be calculated and plotted not only genome wide (as done in Example 3) but also according to copy number (as done in Example 4).

For profiles computed in regions of CN gain, one expects to see an increase in the proportion of short fragments, whereas an increase in the proportion of long fragments is expected for profiles computed in regions of CN loss.

Because of these differences, there is a critical fragment length below which the gain profile is observed on top of the loss profile, and above which the loss profile is observed on top of the gain profile. The neutral profile sits somewhere in between gain and loss profiles.

The separation between fragment length curves within a single sample is quantified according to the following scheme. Below the critical fragment length, the loss profile is subtracted from the gain profile, and the resulting differences are summed together to obtain quantity A. After the critical fragment length, the gain profile is subtracted from the loss profile, and the resulting differences are summed together to obtain quantity B. The separation is the sum of A and B.

In case one of loss or gain profiles is not available, the neutral profile is used in its place. Three formulae can then be used to compute the separation: the loss-gain formula, the loss-neutral formula, and the neutral-gain formula, the names describing which profiles are utilized.

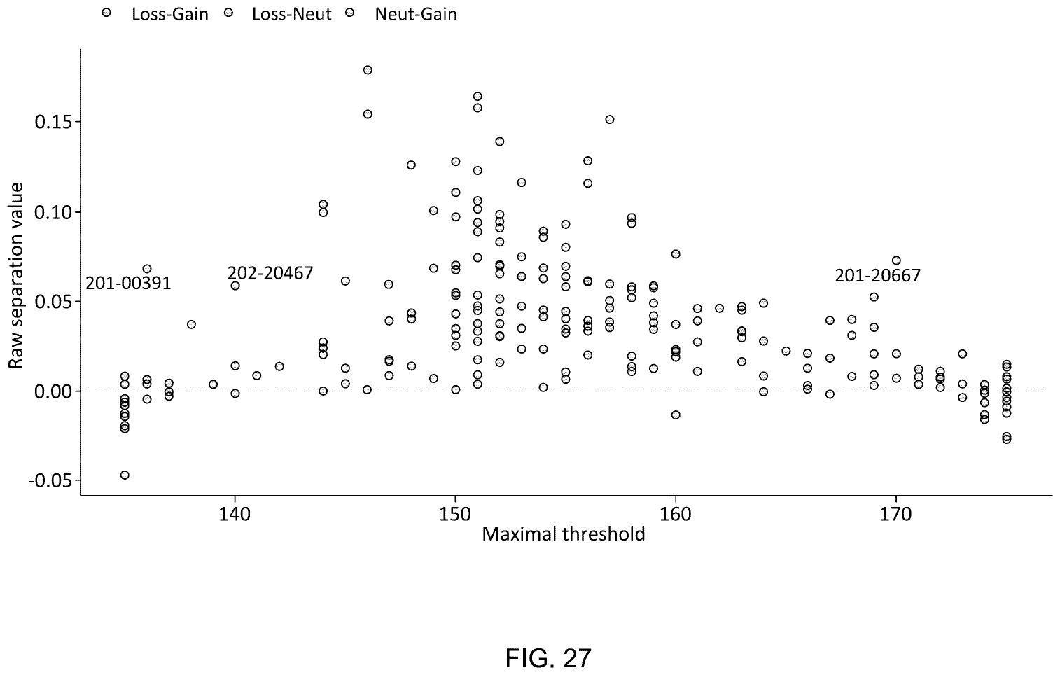

Calculation of the separation value depends on the location of the critical fragment length. Various approaches can be considered to deal with this unknown: a single threshold can be used for all samples, a central interval including the critical fragment length can be ignored, or a threshold can be optimized for each sample (FIG. 27).

Fragment length profiles supported by too few reads may be unreliable. For this reason, one can remove profiles with a read count under a certain threshold, including but not limited to 100,000 reads, 200,000 reads, 500,000 reads, 1,000,000 reads.

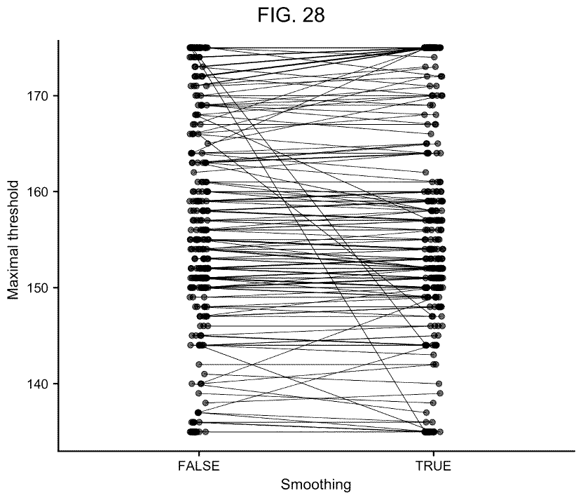

Additionally, the profiles can be smoothed using splines. This, however, does not affect the separation value. The threshold providing the largest separation also remains stable (FIG. 28), except in a few cases without actual separation between the fragment length profiles.

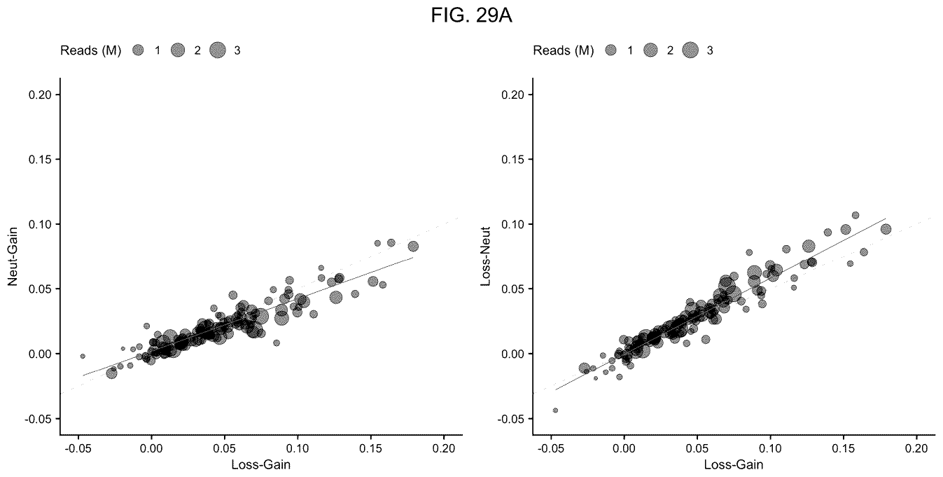

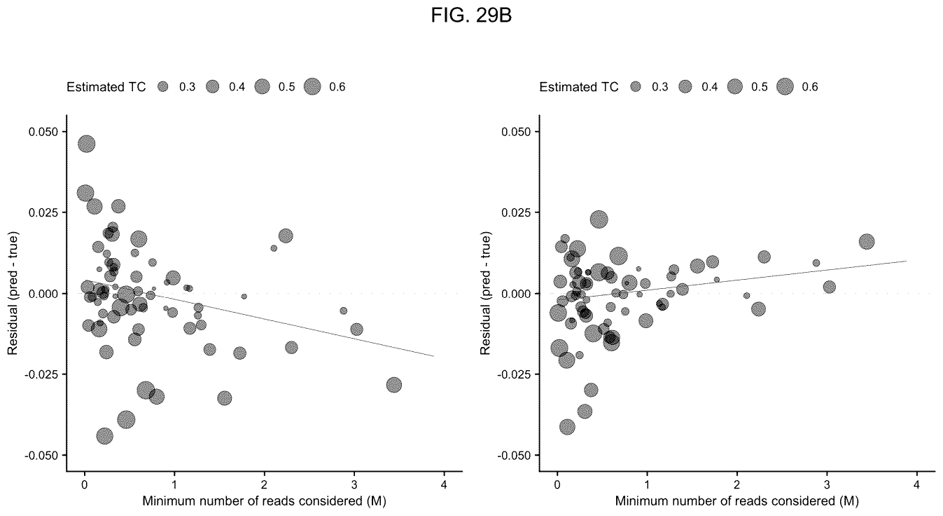

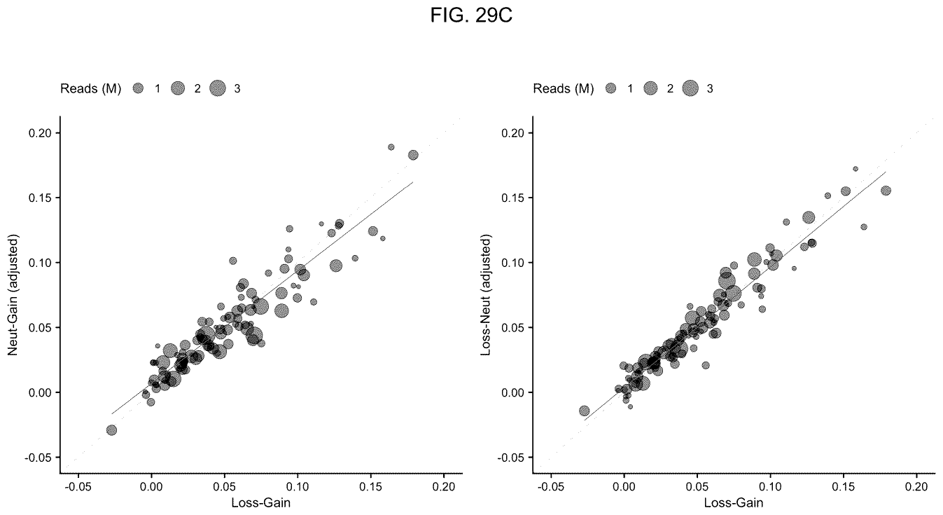

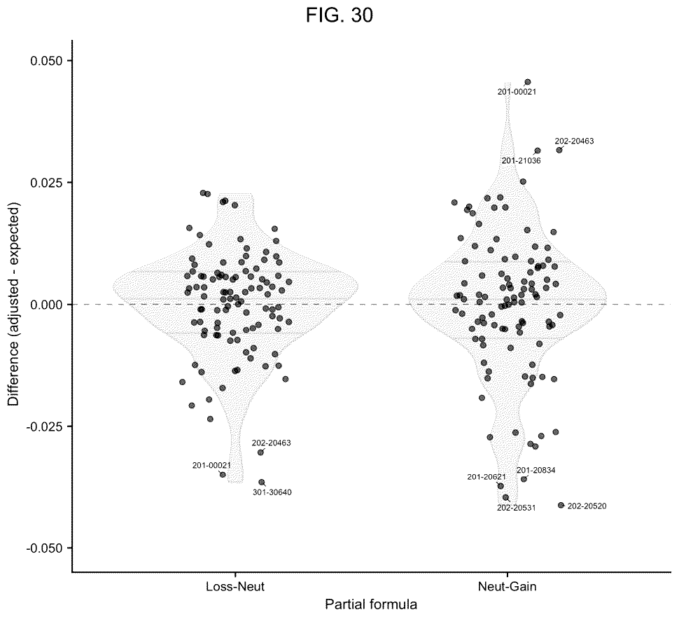

Because the neutral profile lies between the loss and gain profiles, separation values computed using the loss-neutral or neutral-gain formulae are smaller than those computed using the loss-gain formula. Samples with all three levels available were analyzed to quantify this effect (FIG. 29A). The resulting linear relationships could be leveraged to obtain a simple linear correction.

The residuals of this correction did not show strong correlations with estimated tumor content of the minimum number of reads supporting a fragment length profile (FIG. 29B). The largest residuals, however, were observed for profiles supported by a small number of reads.

Adjusted separation values of the loss-neutral and neutral-gain formulae, after application of read filter of 200,000 reads, closely matched the separation values of the loss-gain formula (FIG. 29C). This demonstrated that all three scenarios could be rendered equivalent and analyzed together (FIG. 30).

### Example 7

### Fragmentomics of Hematological Cancer

The following example demonstrates a fragmentomics analysis of hematological cancer samples. Following the observation that a hematological cancer sample with a clean CN profile displayed no separation in terms of fragment length curves, other confirmed hematological cancer cases were tested to check whether this is a feature of that malignancy. If it is, this would be another approach to classify a sample as hematological cancer.

112 samples of confirmed lymphoma were reviewed and categorized for their copy number variations (CNVs) as loss, neutral, or gain.

Fragment length curves were plotted for each CN group, with the expectation that the fragment length profile does not change despite the differential tumor contributions to each CN level. This seems to be explained by the fact that, in healthy subjects, the majority of cfDNA originates from white blood cells, and so a malignancy of the same tissue would share the same nucleosome organization and DNA fragmentation.

90 out of 112 samples had visible CNVs that could be annotated. In 18/90 cases (20%), separation between the fragmentomics curves was observed. Separation is defined as observing (i) distinct lines on the long side of the main nucleosomal peaks, and (ii) the most tumor-enriched profile (e.g. gain at CN 5) above the most tumor-depleted profile (CN 1) before approximately 150 bp, and the opposite thereafter.

The main analysis considered all CNV-positive clinical validation (CV) samples whose CNVs could be categorized as losses, neutral CN or gains. 245 samples were thus considered. Separation scores were computed using the top-bottom formula, which compares loss and gain curves by default (“full” formula), and resorts to loss-neutral or neutral-gain comparisons otherwise (“partial” formula). CN levels supported by fewer than 200,000 reads were ignored. This left 214 samples. Separation values obtained with the “partial” formula were corrected to be in line with the “full” formula. The regression model that performed the correction did not include an intercept.

Labels (denoting separation, no separation-low tumor content, no separation-high tumor content) were assigned to samples following manual review. Separation between fragment length curves was expected, with the gain curve on top of the neutral one, in turn on top of the loss curve for short fragments and, after a change point around 150 bp (FIG. 27), a similar separation but with the order reversed for long fragments. The labels thus obtained fit well with the separation score computed above.

The identification of samples with no separation despite evidence of high tumor content from CNV data was of interest. After filtering only samples with separation and those without separation but with high tumor content, all possible calling thresholds were considered. It was determined that a threshold at 0.0173 produced the best results (97.7% sensitivity, 98.5% specificity). This analysis was done blind to the tumor types.

Considering the two categories of interest, with this threshold there were two false positives (samples with visible separation but a low separation value). Separation in these samples was identified, and samples similar to these were identified in review and removed from the calls: no cancer signal origin (CSO) prediction would be made for these samples on the basis of fragmentomic curves separation.

Before unblinding, clear batch effects that may affect the performance of this method were assessed. A minor upward trend when the sequencing run was considered. However, the 95% confidence interval around the regression line included a horizontal line. This means that the null hypothesis of no batch effect could not be rejected. A similar conclusion was drawn from the age covariate, as well as for gender.

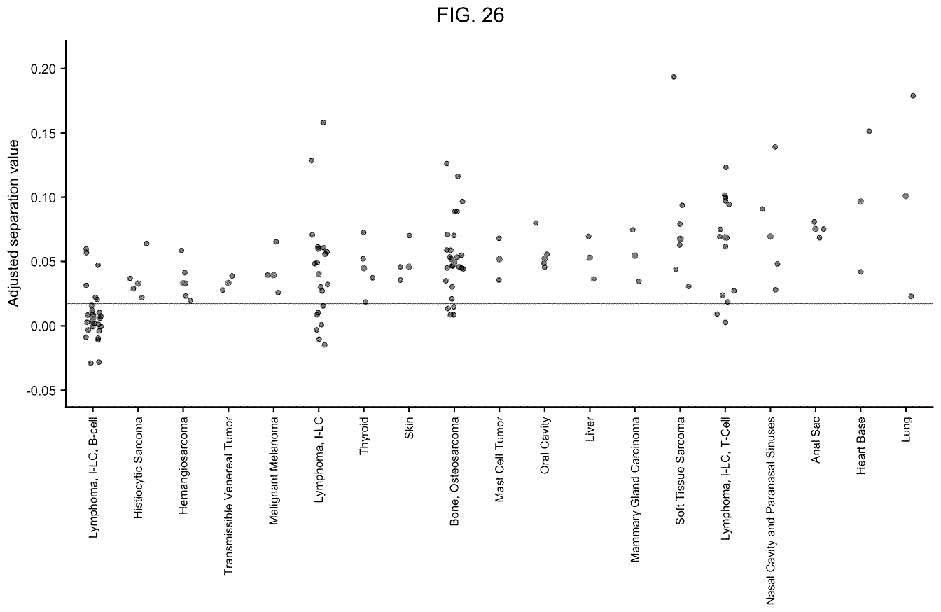

On unblinding, B-cell lymphomas tended to have no separation despite high tumor content, whereas every other cancer type, including T-cell lymphomas, displayed separation of the fragment length curves (FIG. 26). Samples with an adjusted separation value<0.01727873 and labelled no-separation/high-TC after review were the final calls, as shown in Table 4.

TABLE 4

| Cancer type | Count |
|---|---|
| Lymphoma, I-LC, B-cell | 23 |
| Lymphoma, I-LC | 7 |
| Bone, Osteosarcoma | 3 |
| Lymphoma, I-LC, T-Cell | 2 |
| Abdominal Cavity | 1 |
| Hemangiosarcoma | 1 |
| Leukemia, ALL, T-Cell | 1 |
| Leukemia, CLL, T-Cell | 1 |
| Lymphoma, I-LC; Lymphoma, Indolent, B-cell | 1 |
| Mast Cell Tumor; Leukemia, CLL | 1 |
| NA (101-00290) | 1 |
| Urinary Bladder/Urethra; Lymphoma, I-LC | 1 |

The testing set performance was then calculated, as shown in Table 5. The fragmentomics approach required at least two confirmed CN levels (loss and neutral; neutral and gain; or loss and gain), supported by at least 200 k reads. Samples that did not meet these criteria cannot receive a separation score. The current heme prediction in the commercial OncoK9 test is based on the CN profile which has been previously shown to have features associated with hematological cancer (https://pubmed.ncbi.nlm.nih.gov/14562028/). The fragmentomics of hematological cancer as described in this example improved the sensitivity as shown in Table 5.

TABLE 5

| | | Current approach + |
|---|---|---|
| Metric | Current heme prediction | fragmentomics update |
| Sensitivity | 39.58% | 47.92% |
| | (CI: 29.74-50.08) | (CI: 37.61-58.36) |
| PPV | 92.68% | 88.46% |
| | (CI: 80.08-98.46) | (CI: 76.56-95.65) |

### Example 8

### Fragmentomics by Chromosome

The following example demonstrates the use of fragmentomics by chromosome to detect cancer signal in a cfDNA sample.

One way to increase sensitivity for cancer detection is to increase the signal from the tumor. In blood, tumor content is usually small. Chromosomal gains result in an increase of tumor DNA, while losses have the opposite effect. The standard fragmentomics analysis looks at all reads genome-wide, diluting signal from gained regions with signal from copy-number neutral regions and - worse - signal from chromosomal losses.

This analysis looked at fragmentomics by chromosome, expecting to see and leverage differences between individual chromosomes. Enabling this analysis is the knowledge that only about 100,000 fragments are needed to delineate a fragment length profile.

Chromosomes were compared to one another within each sample; for each pair of chromosomes, the KL divergence was compared between their fragment length distributions. When a tumor was present and there were CNAs, chromosomes that are copy-number altered displayed consistently higher deviations from copy-number neutral (CNN) chromosomes.

By understanding what KL divergence values can be expected in healthy samples, a calling threshold can be established to identify cancer-positive samples without needing to compare test samples to normal samples.

To identify a calling threshold, the pairwise comparisons between chromosomes in euploid normal samples were considered. A look at the average KL divergence per chromosome in normal samples revealed that baseline values were heterogeneous, with a pattern inversely related to chromosome length. Longer chromosomes obtained more reads, and more reads produced smoother fragment length profiles, less susceptible to noise and artifactual KL increases.

Additionally, chromosome 9 was an outlier. While it is only approximately as long as chromosomes 14 and 16, its average KL divergence was much higher than expected, probably due to the high GC-content of its sequence.

To address the artifactual increase in KL divergence observed at certain chromosomes (chromosome 9 and short chromosomes) one could correct the observed KL values by a single factor, be it chromosome length or the average KL across samples. Alternatively, one could model the relationship between the average KL and the number of reads.

Focusing on autosomes, each of the KL curves was modelled as a hyperbola with the formula:

y=a/(x+b).

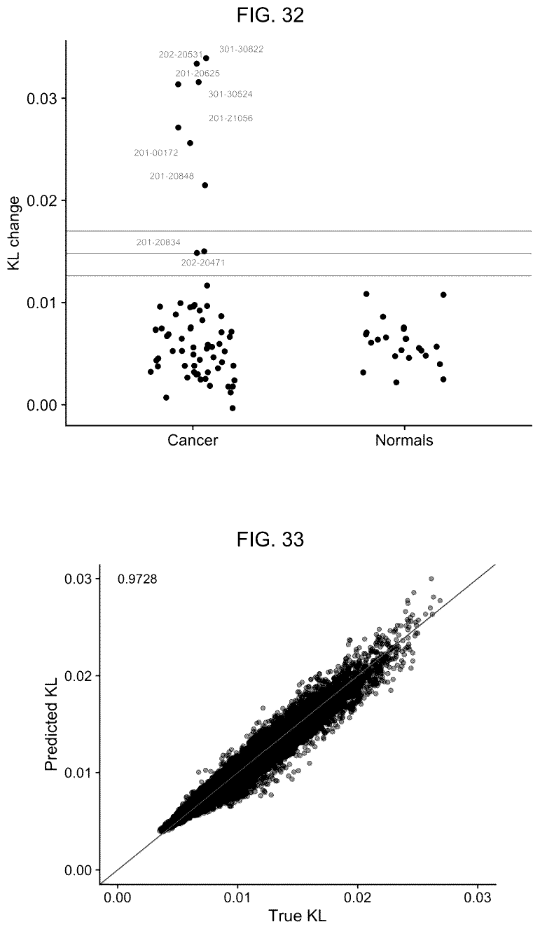

This function was then fitted per chromosome using the least squares. Models such as FIG. 32 were obtained, where the observed chromosome-level KL divergence values were normalized as a function of the number of reads mapped to each chromosome.

This correction eliminated both the KL gradient due to the number of reads and chromosome-specific artifacts. While this correction focused on the number of reads, it did so per chromosome. This, implicitly, accounted for the differences in GC content between chromosomes. If needed, a more sophisticated correction would jointly account for the number of reads in a region and the GC content of the region.

To call samples positive according to this fragmentomics by chromosome approach, chromosomes with large normalized average KL values are identified. If these values are above a certain threshold, the sample is deemed cancer-positive.

The threshold can be defined in various ways: it can be a number of standard deviations above the mean of the normal samples, a threshold that optimizes accuracy on the dataset, etc.

As a start, the adjusted average KL values by chromosome were considered, and a single threshold was defined using mean and standard deviation (SD). The results, compared to the baseline of genome-wide fragmentomics, are shown in Table 6.

TABLE 6

| Method | TP | FP | TN | FN | Sensitivity | Specificity |
|---|---|---|---|---|---|---|
| Baseline | 14 | 0 | 23 | 51 | 0.2154 | 1.0000 |
| Single threshold (3 SDs) | 21 | 3 | 20 | 44 | 0.3231 | 0.8696 |
| Single threshold (4 SDs) | 15 | 0 | 23 | 50 | 0.2308 | 1.0000 |
| Chromosome-specific (3 SDs) | 22 | 2 | 21 | 43 | 0.3385 | 0.9130 |
| Chromosome-specific (4 SDs) | 17 | 0 | 23 | 48 | 0.2615 | 1.0000 |

Despite the small number of normal samples, on this dataset the performance of this fragmentomics by chromosome method was superior to the genome wide baseline.

Correction by the model described above acted on averages per chromosome, not single values. As such, cancer signal resulting in small deviations might be diluted as the average is computed. Instead, considering each pairwise comparison of chromosomes in a sample can prove more sensitive.

To enable this approach, the normalization of the KL values can be expanded to pairs of chromosomes. As before, each chromosome is described by a hyperbola with chromosome-specific parameters. The KL divergence of a pairwise comparison is now modelled as the sum of two chromosome-specific hyperbolae:

y=a/(x1+b)+c/(x2+d).

The chromosome-specific parameters were learnt by Markov chain Monte Carlo using as data the pairwise KL divergence values for a set of normal samples. The correlation between predicted and observed KL values as a function of the number of reads mapped to the chromosomes was 0.9728 (FIG. 33).

Normalization of the pairwise divergence values in test samples was effective at removing chromosome-specific and chromosome length artifacts.

Calling threshold were established for each pairwise comparison. Options for choosing thresholds include, but are not limited to, selecting a number of standard deviations away from the mean of the control samples, or modelling the control values with a probability distribution and selecting a percentile from this distribution, as shown in Table 7.

TABLE 7

| Method | TP | FP | TN | FN | Sensitivity | Specificity |
|---|---|---|---|---|---|---|
| CNV | 27 | 0 | 23 | 37 | 42.19% | 100.00% |
| Fragmentomics | 14 | 0 | 23 | 51 | 21.54% | 100.00% |
| CNV + Frag | 31 | 0 | 23 | 34 | 47.69% | 100.00% |
| Single 3 SDs | 47 | 12 | 11 | 18 | 72.31% | 47.83% |
| Single 4 SDs | 39 | 7 | 16 | 26 | 60.00% | 69.57% |
| Single 5 SDs | 32 | 5 | 18 | 33 | 49.23% | 78.26% |
| Cell 3 SDs | 42 | 6 | 17 | 23 | 64.62% | 73.91% |
| CNV + Cell 3 SDs | 49 | 6 | 17 | 16 | 75.38% | 73.91% |
| Cell 4 SDs | 33 | 0 | 23 | 32 | 50.77% | 100.00% |
| CNV + Cell 4 SDs | 41 | 0 | 23 | 24 | 63.08% | 100.00% |
| Cell 5 SDs | 24 | 0 | 23 | 41 | 36.92% | 100.00% |
| CNV + Cell 5 SDs | 34 | 0 | 23 | 31 | 52.31% | 100.00% |

Genome wide fragmentomics compares the genome wide fragment length profile of a test sample to the profile of a set of normal samples. An alternative fragmentomics by chromosome approach is to apply the same genome wide methodology but to individual chromosomes in a test sample, and thus compare them to an external reference. The test sample's status is then established by taking, for example, the most extreme chromosome-level result, or the largest change compared to the genome wide approach.

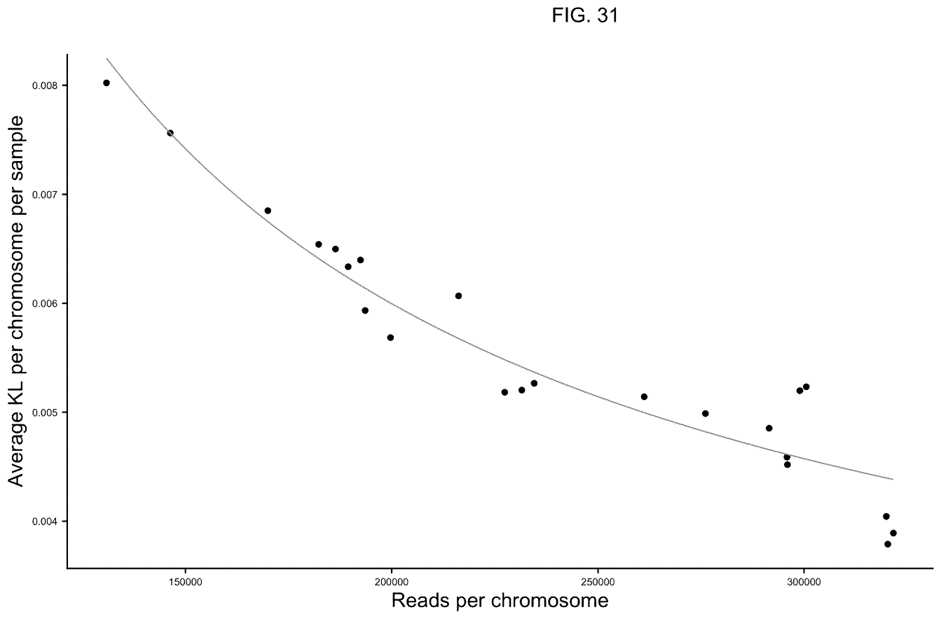

Shown in FIG. 31 and Table 8 are the changes in KL divergence and lines at 3, 4, and 5 SDs away from the mean of the normal samples. Nine samples were called with a threshold at 3 SDs.

TABLE 8

| Method | TP | FP | TN | FN | Sensitivity | Specificity |
|---|---|---|---|---|---|---|
| Genome-wide (baseline) | 14 | 0 | 24 | 54 | 0.2059 | 1.0000 |
| Maximum by chromosome | 15 | 0 | 24 | 53 | 0.2206 | 1.0000 |
| Difference in KL (delta) 3 SD | 9 | 0 | 23 | 56 | 0.1385 | 1.0000 |
| Genome wide or delta 3 SD | 18 | 0 | 23 | 47 | 0.2769 | 1.0000 |
| Maximum or delta 3 SD | 19 | 0 | 23 | 46 | 0.2923 | 1.0000 |

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose, including the disclosures specifically referenced herein. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this invention has been disclosed in the context of certain embodiments and examples, those skilled in the art will understand that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes or embodiments of the disclosed invention. Thus, it is intended that the scope of the present invention herein disclosed should not be limited by the particular disclosed embodiments described above.

It should be understood, however, that this detailed description, while indicating preferred embodiments of the invention, is given by way of illustration only, since various changes and modifications within the spirit and scope of the invention will become apparent to those skilled in the art.

The terminology used in the description presented herein is not intended to be interpreted in any limited or restrictive manner. Rather, the terminology is simply being utilized in conjunction with a detailed description of embodiments of the systems, methods, and related components. Furthermore, embodiments may comprise several novel features, no single one of which is solely responsible for its desirable attributes or is believed to be essential to practicing the inventions herein described.

What is claimed is:

1. A method of detecting a cancer or tumor in a subject, the method comprising:
isolating a circulating cell free DNA (cfDNA) sample from the subject;
sequencing the cfDNA sample to measuring one or more fragment size distribution;
comparing the one or more fragment size distribution to a second fragment size distribution, wherein the second fragment size distribution is obtained from one or more control subject; and
determining the presence of the cancer or tumor based upon the comparisons of the two distributions.

2. The method of claim 1, wherein the one or more subjects comprise the same subject or one or more healthy subjects.

3. The method of claim 1, wherein the sequencing of the cfDNA sample is whole genome sequencing or next generation sequencing.

4. The method of claim 1, further comprising creating a model of the one or more fragment size distribution.

5. The method of claim 4, wherein the model of the one or more fragment size distribution is a statistical model.

6. The method of claim 4, wherein the model of the one or more fragment size distribution is obtained from one or more features extracted from the one or more fragment size distribution.

7. The method of claim 6, wherein the one or more features comprise median, mean, area under the curve (AUC), amplitude of oscillations, variance, standard deviations, length intervals, or a combination thereof.

8. The method of claim 6, further comprising classifying samples as tumor or normal based on the one or more features.

9. The method of claim 4, wherein the model of the second fragment size distribution is a mixture model.

10. The method of claim 1, wherein comparing the one or more fragment size distribution to the second fragment size distribution is performed through a distance or similarity measure.

11. The method of claim 10, wherein the distance or similarity measure is a KL divergence.

12. The method of claim 1, wherein the one or more fragment size distribution is calculated from at least one of length or sequence of cfDNA fragments in the sample.

13. The method of claim 1, wherein the second fragment size distribution is a baseline fragment size distribution.

14. The method of claim 1, wherein the cfDNA sample is isolated from the blood of the subject.

15. The method of claim 14, wherein the blood of the subject further comprises circulating tumor DNA (ctDNA).

16. The method of claim 1, further comprising ligating adapters to the isolated cfDNA and using a universal primer to target the adapters to generate amplified fragments.

17. The method of claim 16, wherein the one or more fragment size distribution is measuring by determining a number and distribution of amplified fragment sizes using whole genome sequencing or next generation sequencing.

18. The method of claim 16, wherein comparing the one or more fragment size distribution to the second fragment size distribution is performed by comparing the number and distribution of the amplified fragment sizes to one or more healthy subjects or to the same subject to determine if the number and distribution of the amplified fragment sizes in the subject differs from the number and distribution of the amplified fragment sizes in the one or more healthy subjects.

19. The method of claim 16, wherein the universal primer further comprises a sequence specific primer.

20. The method of claim 1, wherein a statistically significant difference between the one or more fragment size distribution in the subject and the second fragment size distribution in the one or more healthy subjects indicates the presence of a cancer or tumor.
